(19) United States
(12) Patent Application Publication
(10) Pub. No.: US 2008/0200510 A1
(43) Pub. Date: Aug. 21, 2008

(54) Combination of Organic Compounds

(76) Inventor: Smiljana Milosavljevic-Ristic, Aesch (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/995,251

(22) PCT Filed: Jul. 11, 2006
(86) PCT No.: PCT/EP06/64117

§ 371 Date: Jan. 10, 2008

Related U.S. Application Data
(60) Provisional application No. 60/698,304, filed on Jul. 12, 2005.

(51) Int. Cl. A61K 31/4453; A61K 31/40

(52) U.S. Cl. 514/326; 514/423

(57) ABSTRACT

The present invention relates to a combination, such as a combined preparation or pharmaceutical composition, respectively, comprising of a DPP IV inhibitor or a pharmaceutically acceptable salt thereof and comprising at least one CB1 antagonist, or a pharmaceutically acceptable salt thereof.

The present invention furthermore relates to the use of such a combination for the prevention of, delay of progression of, treatment of diseases and disorders that may be inhibited by DPP IV inhibition, appetency disorders or substance abuse disorders.

This invention relates to a pharmaceutical combination comprising of cannabinoid receptor-1 (CB1) antagonists and a DPP-IV inhibitors, in particular for the prevention, delay of progression or treatment of diseases and disorders that may be inhibited by DPP IV inhibition, appetency disorders or substance abuse disorders.

Obesity and overweight greatly increase the risk of many diseases such as hypertension; type 2 diabetes; dyslipidemia; coronary heart disease; stroke; gallbladder disease; osteoarthritis; sleep apnea and other respiratory problems.

Weight loss is desirable in the case of diabetes, obesity and overweight individuals. Weight loss can help to prevent many of these harmful consequences, particularly with respect to diabetes and cardiovascular disease (CVD). Weight loss may also reduce blood pressure in both overweight hypertensive and non-hypertensive individuals; serum triglycerides levels and increases the beneficial high-density lipoprotein (HDL)-form of cholesterol. Weight loss also generally reduces somewhat the total serum cholesterol and low-density lipoprotein (LDL)-cholesterol levels. Weight loss may also reduce blood glucose levels in overweight and obese persons.

Weight loss, and hypocaloric diets, are also a primary goals for the control of plasma glucose levels in the treatment of type 2 diabetes. Thus appetite control and weight loss are desirable for the treatment of type 2 diabetes.

While weight loss and appetite control are desirable, it is hard to achieve. Many treatments for the management of appetite, overweight and obesity and the maintenance of weight loss exist. However, recidivism is rampant. Approximately 40 percent of women and 24 percent of men are trying to actively lose weight at any given time. These treatments include, but are not limited to, low-calorie diets and low-fat diets; increased physical exercise; behavioral therapies directed toward reducing food intake; pharmacotherapy; surgery; and combinations of the above.

The pharmacopeia of weight loss is relatively bare. A preferred way to reduce body weight is to reduce the appetite for foods and caloric beverages. Drugs such as sibutramine, dexfenfluramine, orlistat, phenylpropanolamine, phenteramine, or fenfluramine can facilitate weight loss in obese adults when used for prolonged periods. In general, however, the safety of long-term administration of pharmaco-therapeutic weight loss agents is unknown. For instance, recently due to concerns about valvular heart disease observed in patients, fenfluramine and dexfenfluramine have been withdrawn from the market. In the face of the slim pharmacopeia and the high prevalence of obesity and overweight, there is a need for new pharmaceutical methods and compositions to promote and maintain weight loss, for the treatment or prevention of diabetes, obesity, appetency disorders or substance abuse disorders.

It has now been found that a combination comprising at least one CB1 antagonist e.g., as defined below, and a DPP-IV inhibitor as co-agent, e.g., as defined below, has a beneficial effect and is useful in the treatment of obesity, appetency disorders, substance abuse disorders or conditions/disorders that might be treated by DPP-IV inhibition.

Thus, the present invention relates to combinations, such as a combined preparation or pharmaceutical composition, respectively, comprising;
i) a DPP IV inhibitor or a pharmaceutically acceptable salt thereof, and
ii) at least one CB1 antagonist, or a pharmaceutically acceptable salt thereof.

Preferably the present invention relates to a combination (pharmaceutical combination), such as a combined preparation or pharmaceutical composition, respectively, comprising;
i) a DPP IV inhibitor or a pharmaceutically acceptable salt thereof, and
ii) at least one CB1 antagonist, or a pharmaceutically acceptable salt thereof, and at least one additional pharmaceutically acceptable carrier.

Preferably the combination is a pharmaceutical composition or a combined pharmaceutical preparation.

In this pharmaceutical composition, the combination partners (i) and (ii) can be administered together, one after the other or separately in one combined unit dosage form or in two separate unit dosage forms. The unit dosage form may also be a fixed combination.

The term “at least one therapeutic agent” shall mean that in addition to the DPP IV inhibitor one or more, for example two, furthermore three, active ingredients as specified according to the present invention can be combined. Preferably one or two CB1 inhibitors.

The term “DPP-IV” as used herein is intended to mean dipeptidyl peptidase IV, also known as CD26. DPP-IV, a serine protease belonging to the group of post-proline/alanine cleaving amino-dipeptidases, specifically removes the two N-terminal amino acids from proteins having proline or alanine in position 2. DPP-IV can be used in the control of glucose metabolism because its substrates include the insulinotropic hormones glucagon like peptide-1 (GLP-1) and gastric inhibitory peptide (GIP). GLP-1 and GIP are active only in their intact forms; removal of their two N-terminal amino acids inactivates them.

In vivo administration of synthetic inhibitors of DPP-IV prevents N-terminal degradation of GLP-1 and GIP, resulting in higher plasma concentrations of these hormones, increased insulin secretion and, therefore, improved glucose tolerance.

The term “DPP-IV inhibitor” is intended to indicate a molecule that exhibits inhibition of the enzymatic activity of DPP-IV and functionally related enzymes, such as from 1-100% or 20-80% inhibition, and specially preserves the action of substrate molecules, including but not limited to GLP-1, GIP, peptide histidine methionine, substance P, neuropeptide Y, and other molecules typically containing alanine or proline residues in the second amino terminal position. Treatment with DPP-IV inhibitors prolongs the duration of action of peptide substrates and increases levels of their intact, undegraded forms leading to a spectrum of biological activities relevant to the disclosed invention.

For that purpose, chemical compounds are tested for their ability to inhibit the enzyme activity of purified CD26/DPP-IV. Briefly, the activity of CD26/DPP-IV is measured in vitro by its ability to cleave the synthetic substrate Gly-Pro-p-nitroanilide (Gly-Pro-pNA). Cleavage of Gly-Pro-pNA by DPP-IV liberates the product p-nitroanilide (pNA), whose rate of appearance is directly proportional to the enzyme activity. Inhibition of the enzyme activity by specific enzyme inhibitors slows down the generation of pNA. Stronger interaction between an inhibitor and the enzyme results in a slower rate of generation of pNA. Thus, the degree of inhibition of the rate of accumulation of pNA is a direct measure of the strength of enzyme inhibition. The accumulation of pNA is measured spectrophotometrically. The inhibition constant, Ki, for each compound is determined by incubating fixed amounts of enzyme with several different concentrations of inhibitor and substrate.

In the present context “a DPP-IV inhibitor” is also intended to comprise active metabolites and prodrugs thereof, such as active metabolites and prodrugs of DPP-IV inhibitors. An active “metabolite” is an active derivative of a DPP-IV inhibitor produced when the DPP-IV inhibitor is metabolized. A “prodrug” is a compound that is either metabolized to a DPP-IV inhibitor or is metabolized to the same metabolite(s) as a DPP-IV inhibitor.

DPP-IV inhibitors are known in the art. For example, DPP-IV inhibitors are in each case generically and specifically disclosed e.g. in WO 98/19998, DE19616 486 A1, WO 00/34241, WO 95/15309, WO 01/72290, WO01/52825, WO 9310127, WO 9925719, WO 9938501, WO 9946272, WO 9967278 and WO 9967279.

Preferred DPP-IV inhibitors are described in the following patent applications; WO 02053548 especially compounds 1001 to 1293 and examples 1 to 124, WO 02067918 especially compounds 1000 to 1278 and 2001 to 2159, WO 02066627 especially the described examples, WO 02/068420 especially all the compounds specifically listed in the examples I to LXIII and the described corresponding analogues, even preferred compounds are 2(28), 2(88), 2(119), 2(136) described in the table reporting IC50, WO 02083128 especially examples 1 to 13, US 2003096846 especially the specifically described compounds, WO 2004/037181 especially examples 1 to 33, WO 0168603 especially compounds of examples 1 to 109, EP1258480 especially compounds of examples 1 to 60, WO 0181337 especially examples 1 to 118, WO 02083109 especially examples 1A to 1D, WO 030003250 especially compounds of examples 1 to 166, most preferably 1 to 8, WO 03035067 especially the compounds described in the examples, WO 03/035057 especially the compounds described in the examples, US2003216450 especially examples 1 to 450, WO 99/46272 especially compounds of claims 12, 14, 15 and 17, WO 0197808 especially compounds of claim 2, WO 03002553 especially compounds of examples 1 to 33, WO 01/34594 especially the compounds described in the examples 1 to 4, WO 02051836 especially examples 1 to 712, EP1245568 especially examples 1 to 7, EP1258476 especially examples 1 to 32, US 2003087950 especially the described examples, WO 02/076450 especially examples 1 to 128, WO 03000180 especially examples 1 to 162, WO 03000181 especially examples 1 to 66, WO 03004498 especially examples 1 to 33, WO 0302942 especially examples 1 to 68, U.S. Pat. No. 6,482,844 especially the described examples, WO 0155105 especially the compounds listed in the examples 1 and 2, WO 0202560 especially examples 1 to 166, WO 03004496 especially examples 1 to 103, WO 03/024965 especially examples 1 to 54, WO 0303727 especially examples 1 to 209, WO 0368757 especially examples 1 to 88, WO 03074500 especially examples 1 to 72, examples 4.1 to 4.23, examples 5.1 to 5.10, examples 6.1 to 6.30, examples 7.1 to 7.23, examples 8.1 to 8.10, examples 9.1 to 9.30, WO 02038541 especially examples 1 to 53, WO 02062764 especially examples 1 to 293, preferably the compound of example 95 (2-{{3-(Aminomethyl)-4-butoxy-2-neopentyl-1-oxo-1,2 dihydro-6-isoquinolinyl}oxy}acetamide hydrochloride), WO 02308090 especially examples 1-1 to 1-109, examples 2-1 to 2-9, example 3, examples 4-1 to 4-19, examples 5-1 to 5-39, examples 6-1 to 6-4, examples 7-1 to 7-10, examples 8-1 to 8-8, examples 7-1 to 7-7 of page 90, examples 8-1 to 8-59 of pages 91 to 95, examples 9-1 to 9-33, examples 10-1 to 10-20, US 2003225102 especially compounds 1 to 115, compounds of examples 1 to 121, preferably compounds a) to z), aa) to az), ba) to bz), ca) to cz) and da) to dk), WO 0214271 especially examples 1 to 320 and US 2003096857 and WO 2004/052850 especially the specifically described compounds such as examples 1 to 42 and compounds of claim 1, DE 102 56 264 A1 especially the described compounds such as examples 1 to 181 and the compounds of claim 5, WO 04/076433 especially the compounds specifically described, such as listed in table A, preferably the compounds listed in table B, preferably compounds I to XXXXVII, or compounds of claims 6 to 49, WO 04/071454 especially the specifically described compounds e.g. compounds 1 to 53 or compounds of tables Ia to If, or compounds of claims 2 to 55, WO 02/068420 especially the compounds specifically described, such as the compounds I to LXIII or Beispiele I and analogues 1 to 140 or Beispiele 2 and analogues 1 to 174 or Beispiele 3 and analogues 1, or Beispiele 4 to 5, or Beispiele 6 and analogues 1 to 5, or Beispiele 7 and analogues 1-3, or Beispiele 8 and analogue 1, or Beispiele 9, or Beispiele 10 and analogues 1 to 531 even preferred are compounds of claim 13, WO 03/000250 especially the compounds specifically described, such as the compounds 1 to 166, preferably compounds of examples 1 to 9, WO 03/024942 especially the compounds specifically described, such compounds 1 to 59, compounds of table 1 (1 to 68), compounds of claims 6, 7, 8, 9, WO 03024965024942 especially the compounds specifically described, such compounds 1 to 54, Wo03002593 especially the compounds specifically described, such compounds table 1 or of claims 2 to 15, WO03037327 especially the compounds specifically described, such compounds of examples 1 to 209 WO 03/000250 especially the compounds specifically described, such as the compounds 1 to 166, preferably compounds of examples 1 to 9, WO 03/024942 especially the compounds specifically described, such compounds 1 to 59, compounds of table 1 (1 to 68), compounds of claims 6, 7, 8, 9, WO 03024965024942 especially the compounds specifically described, such compounds 1 to 54, Wo03002593 especially the compounds specifically described, such compounds table 1 or of claims 2 to 15, WO03037327 especially the compounds specifically described, such compounds of examples 1 to 209, WO0238541, WO0230890.

WO 03/000250 especially the compounds specifically described, such as the compounds 1 to 166, preferably compounds of examples 1 to 9, WO 03/024942 especially the compounds specifically described, such compounds 1 to 59, compounds of table 1 (1 to 68), compounds of claims 6, 7, 8, 9, WO 03024965 especially the compounds specifically described, such compounds 1 to 54, WO 03002593 especially the compounds specifically described, such compounds table 1 or of claims 2 to 15, WO03037327 especially the compounds specifically described, such compounds of examples 1 to 209, WO0238541 especially the compounds specifically described, such compounds of examples 1 to 53, WO 03/002531 especially the compounds specifically described preferably the compounds listed on page 9 to 13, most preferably the compounds of examples 1 to 46 and even preferred compound of example 9, U.S. Pat. No. 6,395,767 preferably compound of examples 1 to 109 most preferably compound of example 60, U.S. application Ser. No. 09/788,173 filed Feb. 16, 2001 (attorney file LA50) especially the described examples, WO99/38501 especially the described examples, WO99/46272 especially the described examples and DE19616 486 A1 especially val-pyr, val-thiazolidide, isoleucyl-thiazolidide, isoleucyl-pyrrolidide, and fumar salts of isoleucyl-thiazolidide and isoleucyl-pyrrolidide.

Further preferred DPP-IV inhibitors include the specific examples disclosed in U.S. Pat. No. 6,124,305 and U.S. Pat. No. 6,107,317, International Patent Applications, Publication Numbers WO 95153 09 and WO 9818763.

In each case in particular in the compound claims and the final products of the working examples, the subject matter of the final products, the pharmaceutical preparations and the claims are hereby incorporated into the present application by reference to these publications.

Published patent application WO 9819998 discloses N-(N′-substituted glycyl)-2-cyano pyrrolidines, in particular 1-[2-[5-Cyanopyridin-2-yl]amino]-ethylamino]acetyl-2-cyano-(S)-pyrrolidine (NVP-DPP728).

Published patent application WO 0034241 and published patent U.S. Pat. No. 6,110,949 disclose N-substituted adamantyl-amino-acetyl-2-cyano pyrrolidines and W (substituted glycyl)-4-cyano pyrrolidines respectively. DPP-IV inhibitors of interest are specially those cited in claims 1 to 4. In particular these applications describe the compound 1-[[(3-Hydroxy-1-adamantyl) amino]acetyl]-2-cyano-(S)-pyrrolidine (also known as LAF237 or vildagliptin).

Published patent application WO 9515309 discloses amino acid 2-cyanopyrrolidine amides as inhibitors of DPP-IV Published patent application WO 9529691 discloses peptidyl derivates of diesters of alpha-aminoalkylphosphonic acids, particularly those with proline or related structures. DPP-IV inhibitors of interest are specially those cited in Table 1 to 8.

In WO 01/72290 DPP-IV inhibitors of interest are specially those cited in example 1 and claims 1, 4, and 6.

WO01/52825 specially discloses (S)-1-{2-[5-cyanopyridin-2yl)amino]ethyl-aminoacetyl)-2-cyano-pyrrolidine or (S)-1-[(3-hydroxy-1-adamantyl)amino]acetyl-2-cyano-pyrrolidine.

Published patent application WO 9310127 discloses proline boronic esters useful as DPP-IV inhibitors. DPP-IV inhibitors of interest are specially those cited in examples 1 to 19.

Published patent application WO 9925719 discloses sulphostin, a DPP-IV inhibitor prepared by culturing a Streptomyces microorganism.

Published patent application WO 9938501 discloses N-substituted 4-8 membered heterocyclic rings. DPP-IV inhibitors of interest are specially those cited in claims 15 to 20.

Published patent application WO 9946272 discloses phosphoric compounds as inhibitors of DPP-IV. DPP-IV inhibitors of interest are specially those cited in claims 1 to 23.

Published patent applications WO 9967278 and WO 9967279 disclose DPP-IV prodrugs and inhibitors of the form A-B-C where C is either a stable or unstable inhibitor of DPP-IV.

Other preferred DPP-IV inhibitors are the compounds of formula I, II or III disclosed in the patent application WO 03/057200 on page 14 to 27. Most preferred DPP-IV inhibitors are the compounds specifically described on pages 28 and 29.

Any of the substances disclosed in the above mentioned patent documents, hereby included by reference, are considered potentially useful as DPP-IV inhibitors to be used in carrying out the present invention.

In a further preferred embodiment, the DPP-IV inhibitor is a N-peptidyl-O-aroyl hydroxylamine or a pharmaceutically acceptable salt thereof. Aroyl is, for example, naphthylcarbonyl; or benzoyl which is unsubstituted or mono- or disubstituted, for example, by lower alkoxy, lower alkyl, halogen or, preferably, nitro. The peptidyl moiety comprises preferably two α-amino acids, e.g. glycine, alanine, leucine, phenylalanine, lysine or proline, of which the one attached directly to the hydroxylamine nitrogen atom is preferably proline.

Preferably, the N-peptidyl-O-aroyl hydroxylamine is a compound of formula VII

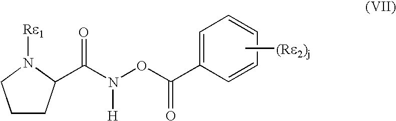

wherein

j is 0, 1 or 2;

Rε₁ represents the side chain of a natural amino acid; and

Rε₂ represents lower alkoxy, lower alkyl, halogen or nitro;

or a pharmaceutically acceptable salt thereof.

In a very preferred embodiment of the invention, the N-peptidyl-O-aroyl hydroxylamine is a compound of formula VIIa

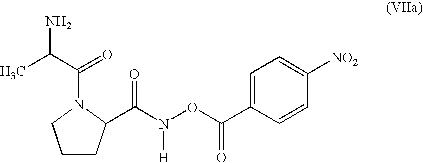

or a pharmaceutically acceptable salt thereof.

N-Peptidyl-O-aroyl hydroxylamines, e.g. of formula VII or VIIa, and their preparation are described by H. U. Demuth et al. in J. Enzyme Inhibition 1988, Vol. 2, pages 129-142, especially on pages 130-132.

Preferred DPP-IV inhibitors are N-substituted adamantyl-amino-acetyl-2-cyano pyrrolidines, N (substituted glycyl)-4-cyano pyrrolidines, N—(N′-substituted glycyl)-2-cyanopyrrolidines, N-aminoacyl thiazolidines, N-aminoacyl pyrrolidines, L-allo-isoleucyl thiazolidine, L-threo-isoleucyl pyrrolidine, and L-allo-isoleucyl pyrrolidine, 1-[2-[(5-cyanopyridin-2-yl)amino]ethylamino]acetyl-2-cyano-(S)-pyrrolidine and pharmaceutical salts thereof.

Preferred DPP-IV inhibitors are those described by Mona Patel and col. (Expert Opinion Investig Drugs. 2003 April;12(4):623-33) on the paragraph 5, especially P32/98, K-364, FE-999011, BDPX, NVP-DDP-728 and others, which publication is hereby incorporated by reference especially the described DPP-IV inhibitors.

Another preferred inhibitor is the compound BMS-477118 disclosed in WO 2001068603 or U.S. Pat. No. 6,395,767 (compound of example 60) also known as is (1S,3S,5S)-2-[(2S)-2-amino-2-(3-hydroxytricyclo[3.3.1.1^{3,7}]dec-1-yl)-1-oxoethyl]-2-azabicyclo[3.1.0]hexane-3-carbonitrile, benzoate (1:1) as depicted in Formula M of the patent application WO 2004/052850 on page 2, and the corresponding free base, (IS,3S,5S)-2-[(2S)-2-amino-2-(3-hydroxy-tricyclo[3.3.1.1^{3,7}]dec-1-yl)-1-oxoethyl]-2-azabicyclo-[3.1.0]hexane-3-carbonitrile (M′) and its monohydrate (M″) as depicted in Formula M of the patent application WO 2004/052850 on page 3. The compound BMS-477118 is also known as saxagliptin.

Another preferred inhibitor is the compound GSK23A disclosed in WO 03/002531 (example 9) also known as (2S,4S)-1-((2R)-2-Amino-3-[(4-methoxybenzyl)sulfonyl]-3-methylbutanoyl)-4-fluoropyrrolidine-2-carbonitrile hydrochloride.

FE-999011 is described in the patent application WO 95/15309 page 14, as compound No. 18.

P32/98 or P3298 (CAS number: 251572-86-8) also known as 3-[(2S,3S)-2-amino-3-methyl-1-oxopentyl]thiazolidine can be used as 3-[(2S,3S)-2-amino-3-methyl-1-oxopentyl]thiazolidine and (2E)-2-butenedioate (2:1) mixture such as shown below

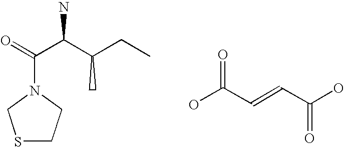

and is described in WO 99/61431 and also in Diabetes 1998, 47, 1253-1258, in the name of Probiodrug, as well as the compound P93/01 described by the same company.

Other very preferred DPP-IV inhibitors of the invention are described in the International patent application WO 02/076450 (especially the examples 1 to 128) and by Wallace T. Ashton (Bioorganic & Medicinal Chemistry Letters 14 (2004) 859-863 ) especially the compound 1 and the compounds listed in the tables 1 and 2. The preferred compound is the compound 21e (table 1) of formula:

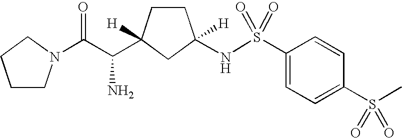

Other preferred DPP-IV inhibitors are described in the patent applications WO 2004/037169 especially those described in the examples 1 to 48 and WO 02/062764 especially the described examples 1 to 293, even preferred are the compounds 3-(aminomethyl)-2-isobuthyl-1-oxo-4-phenyl-1,2-dihydro-6-isoquinolinecarboxamide and 2-{[3-(aminomethyl)-2-isobuthyl-4-phenyl-1-oxo-1,2-dihydro-6-isoquinolyl]oxy}acetamide described on page 7 and also in the patent application WO2004/024184 especially in the reference examples 1 to 4.

Other preferred DPP-IV inhibitors are described in the patent application WO 03/004498 especially examples 1 to 33 and most preferably the compound of the formula

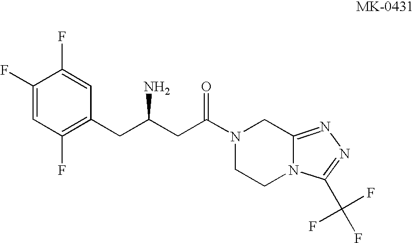

described by the example 7 and also known as MK-0431 or Sitagliptin.

Preferred DPP-IV inhibitors are also described in the patent application WO 2004/037181 especially examples 1 to 33 and most preferably the compounds described in the claims 3 to 5.

Preferred DPP-IV inhibitors are N-substituted adamantyl-amino-acetyl-2-cyano pyrrolidines, N (substituted glycyl)-4-cyano pyrrolidines, N-(N′-substituted glycyl)-2-cyanopyrrolidines, N-aminoacyl thiazolidines, N-aminoacyl pyrrolidines, L-allo-isoleucyl thiazolidine, L-threo-isoleucyl pyrrolidine, and L-allo-isoleucyl pyrrolidine, 1-[2-[(5-cyanopyridin-2-yl) amino]ethylamino]acetyl-2-cyano-(S)-pyrrolidine, MK-431 and pharmaceutical salts thereof.

Most preferred DPP-IV inhibitors are selected from [S]-1-[2-(5-cyano-2-pyridinylamino)ethylamino]acetyl-2-pyrolidine carbonitrile monohydrochloride, (S)-1-[(3-hydroxy-1-adamantyl)amino]acetyl-2-cyano-pyrrolidine and L-threo-isoleucyl thiazolidine (compound code according to Probiodrug: P32/98 as described above), MK-0431, 3-(aminomethyl)-2-isobuthyl-1-oxo-4-phenyl-1,2-dihydro-6-isoquinolinecarboxamide and 2-{[3-(aminomethyl)-2-isobuthyl-4-phenyl-1-oxo-1,2-dihydro-6-isoquinolyl]oxy}acetamide and optionally pharmaceutical salts thereof.

Especially preferred are 1-{2-[(5-cyanopyridin-2-yl)amino]ethylamino}acetyl-2(S)-cyano-pyrrolidine dihydrochloride (DPP728) (also named [S]-1-[2-(5-cyano-2-pyridinylamino)ethylamino]acetyl-2-pyrolidine carbonitrile monohydrochloride), of formula

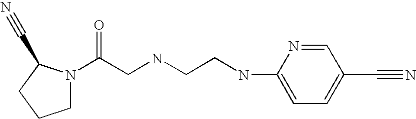

especially the dihydrochloride and monohydrochloride thereof, and 1-[(3-hydroxy-1-adamantyl)amino]acetyl-2-cyano-, (S) (also named (S)-1-[(3-hydroxy-1-adamantyl)amino]acetyl-2-cyano-pyrrolidine, LAF237 or vildagliptin) of formula

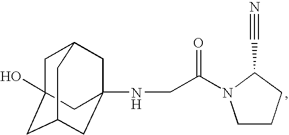

and L-threo-isoleucyl thiazolidine (compound code according to Probiodrug: P32/98 as described above), MK-0431, GSK23A, saxagliptin, 3-(aminomethyl)-2-isobuthyl-1-oxo-4-phenyl-1,2-dihydro-6-isoquinolinecarboxamide and 2-{[3-(aminomethyl)-2-isobuthyl-4-phenyl-1-oxo-1,2-dihydro-6-isoquinolyl]oxy}acetamide and optionally pharmaceutical salts thereof.

DPP728 and vildagliptin are specifically disclosed in Example 3 of WO 98/19998 and Example 1 of WO 00/34241, respectively. The DPP-IV inhibitor P32/98 (see above) is specifically described in Diabetes 1998, 47, 1253-1258. DPP728 and LAF237 can be formulated as described on page 20 of WO 98/19998 or in WO 00/34241 or in the International Patent Application No. EP2005/000400 (application number).

Especially preferred are orally active DPP-IV inhibitors.

Any of the substances disclosed in the above mentioned patent documents or scientific publications, hereby included by reference, are considered potentially useful as DPP-IV inhibitors to be used in carrying out the present invention.

In each case in particular in the compound claims and the final products of the working examples, the subject matter of the final products, the pharmaceutical preparations and the claims are hereby incorporated into the present application by reference to these publications.

An antagonist of the CB1 cannabinoid receptor is a compound which binds to the receptor and lacks any substantial ability to activate the receptor itself. An antagonist can thereby prevent or reduce the functional activation or occupation of the receptor by an agonist such as anandamide when the agonist is present. In some embodiments, the antagonist has an IC₅₀ from about 1 μM to about 1 nM. In other embodiments, the antagonist has an IC₅₀ of from about 0.1 μM to 0.01 μM, 1.0 μM to 0.1 μM, or 0.01 μM to 1 nM. In some embodiments, the antagonist competes with the agonist for binding to a shared binding site on the receptor.

A first group of suitable cannabinoid CB1 receptor antagonists are pyrazole derivatives. Patent applications EP-A-576 357 and EP-A-658 546 describe exemplary pyrazole derivatives which have an affinity for the cannabinoid receptors. More particularly, patent application EP-A-656 354 discloses exemplary pyrazole derivatives and claims N-piperidino-5-(4-chlorophenyl)-1-(2,4-dichlorophenyl)-4-methylpyrazole-3-carboxamide, or SR 141716, and its pharmaceutically acceptable salts, which have a very good affinity for the central cannabinoid receptors. Additional exemplary CB1 receptor antagonists are disclosed in U.S. Pat. No. 5,596,106 which discloses both arylbenzo[b]thiophene and benzo[b]furan compounds to block or inhibit cannabinoid receptors in mammals. Preferably, such a cannabinoid antagonist is selective for the CB1 receptor and has an IC₅₀ for the CB1 receptor which is one-fourth or less than that of the CB2 receptor or, more preferably, is one-tenth or less than the IC₅₀ for the CB2 receptor, or even more preferably, an IC₅₀ with respect to the CB1 receptor which is one-hundredth that for the CB2 receptor. Each of the above references is incorporated by reference in its entirety.

Another representative example is Iodopravadoline (AM-630), which was introduced in 1995. AM-630 is a CB₁ receptor antagonist, but sometimes behaves as a weak partial agonist (Hosohata, K.; Quock, R. M.; Hosohata, Y.; Burkey, T. H.; Makriyannis, A.; Consroe, P.; Roeske, W. R.; Yamamura, H. I. Life Sc. 1997, 61, PL1 15). More recently, researchers from Eli Lilly described arylaroyl substituted benzofurans as selective CB₁ receptor antagonists (e.g. LY-320135) (Felder, C. C.; Joyce, K. E.; Briley, E. J.; Glass, M.; Mackie, K. P.; Fahey, K. J.; Cullinan, G. J.; Hunden, D. C.; Johnson, D. W.; Chaney, M. O.; Koppel, G. A.; Brownstein, M. J. Pharmacol. Exp. Ther. 1998, 284, 291). Recently, 3-alkyl-5,5′-diphenylimidazolidinediones were described as cannabinoid receptor ligands, which were indicated to be cannabinoid antagonists (Kanyonyo, M.; Govaerts, S. J.; Hermans, E.; Poupaert, J. H., Lambert, D. M. Biorg. Med. Chem. Lett. 1999, 9, 2233). Interestingly, many CB₁ receptor antagonists have been reported to behave as inverse agonists in vitro (Landsman, R. S.; Burkey, T. H.; Consroe, P.; Roeske, W. R.; Yamamura, H. I. Eur. J. Pharmacol. 1997, 334, R1). Recent reviews provide a nice overview of the current status in the cannabinoid research area (Mechoulam, R.; Hanus, L.; Fride, E. Prog. Med. Chem. 1998, 35, 199. Lambert, D. M. Curr. Med. Chem. 1999, 6, 635. Mechoulam, R.; Fride, E.; Di Marzo, V. Eur. J. Pharmacol. 1998, 359, 1). From the international patent application WO 01/70700 4,5-dihydro-1H-pyrazole compounds are known which exhibit potent and selective cannabis CB₁-receptor antagonistic activity.

Also useful are the cannabinoid CB1 receptor antagonist compounds of the formula

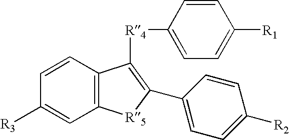

wherein the substituents R₁, R₂, R₃, R₄, and R₅ are defined as recited in U.S. Pat. No. 5,596,106 which is incorporated by reference in its entirety. Related reference U.S. Pat. No. 5,747,524 is also incorporated by reference in its entirety. This reference discloses additional exemplary aryl-benzo[b] thiophene and arylbenzo[b]furan derivatives for use according to the invention.

The cannabinoid antagonists of the following formula are also particularly useful according to the invention:

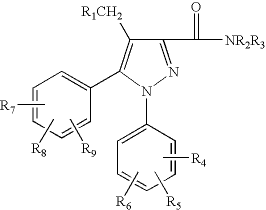

wherein R₁ is hydrogen, a fluorine, a hydroxyl, a (C₁-C₅)alkoxy, a (C₁-C₅)alkylthio, a hydroxy(C₁-C₅)alkoxy, a group —NR₁₀R₁₁, a cyano, a (C₁-C₅)alkylsulfonyl or a (C₁-C₅) alkylsulfinyl;

R₂ and R₃ are a (C₁-C₄)alkyl or, together with the nitrogen atom to which they are bonded, form a saturated or unsaturated 5- to 10-membered heterocyclic radical which is unsubstituted or monosubstituted or polysubstituted by a (C₁-C₃)alkyl or by a (C₁-C₃)alkoxy;

R₄, R₅, R₆, R₇, R₈ and R₉ are each independently hydrogen, a halogen or a trifluoromethyl, and if R₁ is a fluorine, R₄, R₅, R₆, R₇, R₈and/or R₉ can also be a fluoromethyl, with the proviso that at least one of the substituents R₄ or R₇ is other than hydrogen; and

R₁₀ and R₁₁ are each independently hydrogen or a (C₁-C₅)alkyl, or R₁₀ and R₁₁, together with the nitrogen atom to which they are bonded, form a heterocyclic radical selected from pyrrolidin-1-yl, piperidin-1-yl, morpholin-4-yl and piperazin-1-yl, which is unsubstituted or substituted by a (C₁-C₄)alkyl,

and their salts and their solvates.

Other examples of selective CB₁ antagonistic compounds which are useful in the context of the present invention include (without being limited thereto):

1) Diarylpyrazole congeners disclosed by Sanofi as selective CB₁ receptor antagonists, e.g. as representative example the compounds SR-141716A, SR-147778, SR-140098 and rimonabant and related compounds described e.g. in EP 0969835 or EP 1150961(Central mediation of the cannabinoid cue: activity of a selective CB₁ antagonist, SR 141716A Perio A, Rinaldi-Carmona M, Maruani J Behavioural Pharmacology 1996, 7:1 (65-71)); WIN-54461 disclosed by Sanofi-Winthrop (Cannabinoid receptor ligands: Clinical and neuropharmacological considerations relevant to future drug discovery and development. Pertwee R G, Expert Opinion on Investigational Drugs 1996, 5:10 (1245-1253)). N-piperidino-5-(4-chlorophenyl)-1-(2,4-dichlorophenyl)-4-methylpyrazole-3-carboxamide (SR 141616—CAS number: 168273-06-1), its pharmaceutically acceptable salts and their solvates were described for the preparation of drugs useful in the treatment of appetency disorders. SR 141616, (pINN: rimonabant) is represented by the formula:

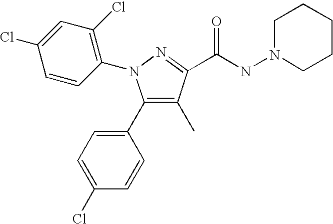

Rimonabant is specifically described in EP-B-656 354 or in an article from M. Rinaldi-Carmona et al. (FEBS Lett., 1994, 350, 240-244). EP1446384 A1 describes new polymorphs of rimonabant, formulation comprising rimonabant are described in WO2003082256, and the use of rimonabant in apetite disorders is described in WO99/00119.

2) Aminoalkylindoles having been disclosed as CB₁ receptor antagonists, e.g. as a representative example the compound Iodopravadoline (AM-630),

3) Aryl-aroyl substituted benzofurans described by Eli Lilly as selective CB₁ receptor antagonists, e.g. LY-320135 (Cannabinoid receptor ligands: Clinical and neuropharmacological considerations relevant to future drug discovery and development. Pertwee R G, Expert Opinion on Investigational Drugs 1996, 5:10 (1245-1253)), 4) Compounds described by Merck & Co, e.g. AM 251 and AM 281 (Conference: 31st Annual Meeting of the Society for Neuroscience, San Diego, USA, 10-15.11.2001), and substituted imidazolyl derivatives disclosed e.g. in U.S. 2003-114495 or WO 03/007887,

5) Azetidine derivatives described by Aventis Pharma e.g. in WO 02/28346 or EP 1328269,

6) CP-55940 from Pfizer Inc. (Comparison of the pharmacology and signal transduction of the human cannabinoid CB1 and CB2 receptors, Felder C C, Joyce K E, Briley E M, Mansouri J, Mackie K, Blond 0, Lai Y, Ma A L, Mitchell R L, Molecular Pharmacology 1995, 48:3 (443)),

6′) The Pfizer compounds described in the patent applications EP1622876, EP1622902, EP1622903, EP162290, EP1622909, EP1638570, EP1594872, EP1592691, EP1558615, EP1556373, EP1572662 especially the specific examples described therein, especially CP-945598.

7) Diaryl-pyrazine-amide derivatives from Astra Zeneca described e.g. in the WO 03/051851,

8) ACPA and ACEA from Med. Coll. Wisconsin (Univ. Aberdeen), (“Effects of AM 251 & AM 281, cannabinoid CB1 antagonists, on palatable food intake in lewis rats” J. Pharmacol. Exp. Ther. 289, No 3, 1427-33, 1999),

9) Pyrazole derivatives described by the University of Conneticut e.g. in the WO 01/29007,

10) HU-210 (International Association for the Study of Pain—Ninth World Congress (Part II) Vienna, Austria, Dickenson A H, Carpenter K, Suzuki R, IDDB MEETING REPORT 1999, August 22-27) and HU-243 (Cannabinoid receptor agonists and antagonists, Barth F, Current Opinion in Therapeutic Patents 1998, 8:3 (301-313)) from Yissum R&D Co Hebrew Univ. of Jerusalem,

11) O-823 from Organix Inc. (Drug development pipeline: O-585, O-823, 0-689, O-1072, nonamines, Organix, Altropane Organix Inc, Company Communication Aug. 10, 1999; IDDb database) and O-2093 from Consiglio Nazionale delle Ricerche (“A structure/activity relationship study on arvanil, endocannabinoid and vanilloid hybrid.”, Marzo D V, Griffin G, Petrocellis L, Brandi I, Bisogno T, Journal of Pharmacology and Experimental Therapeutics 2002, 300:3 (984-991)),

12) 3-Alkyl-5,5′-diphenylimidazolidinediones which were described as cannabinoid receptor ligands,

13) CB₁ antagonistic compounds currently under development by Bayer AG (IDDb database: company communication Feb. 28, 2002).

14) CB1 receptor antagonists are pyrazole derivatives according to Formula (I) Of U.S. Pat. No. 6,028,084 which is incorporated by reference in its entirety.

15) U.S. Pat. No. 6,017,919 discloses another group of suitable cannabinoid receptor antagonists for use according to the invention. These antagonists are of the following general formula:

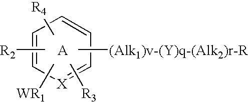

wherein the substituents are as defined in U.S. Pat. No. 6,017,919 which is incorporated herein by reference in its entirety.

16) The CB1 cannabinoid antagonist is a 4,5,dihydro-1H-pyrazole derivative having CB1-antagonist activity as taught in U.S. Pat. No. 5,747,524 and U.S. Patent Application No. 2001/0053788A1 published on Dec. 20, 2001.

17) The CB1 receptor antagonist is a 4,5,dihydro-1H-pyrazole derivative having CB1-antagonistic activity as taught in U.S. Patent Application No. 2001/0053788A1 and particularly disclosed by formula (I) therein. U.S. Patent Application No. 2001/0053788A1 published on Dec. 20, 2001 and is incorporated by reference in its entirety.

18) The CB1 receptor antagonists described in WO2005049615 especially the compounds of example 1 to 8.

19) The CB1 receptor antagonists described in WO2005047285 especially the compounds of example 1 to 99.

20) The CB1 receptor antagonist (4R)-3-(4-chlorophenyl)-4,5-dihydro-N-methyl-4-phenyl-N′-[[4-(trifluoromethyl)phenyl]sulfonyl]-1H-pyrazole-1-carboximidamide (SLV 326—34^{th} Neuroscience, Abs 1009.4, October 2004)

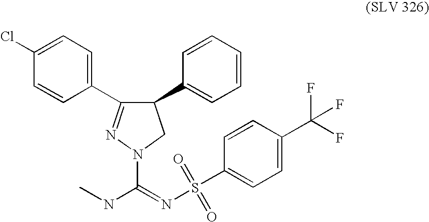

developed by the company Solvay (WO0170700 A1).

Solvay CB1 receptor antagonists are described in the examples of the patent applications WO2005040130 A1, WO2005028456 A1, WO2005020988 A1, WO2004026301 A1, WO2003078413 A1, WO2003027076 A2, WO2003026648 A1, WO2003026647 A1, WO2002076949 A1, WO0170700 A1.

In each case in particular in the compound claims and the final products of the working examples, the subject matter of the final products, the pharmaceutical preparations and the claims are hereby incorporated into the present application by reference to these publications.

The dosage of CB1 antagonist administered will also be generally dependent upon the health of the subject being treated, the extent of treatment desired, the nature and kind of concurrent therapy, if any, and the frequency of treatment and nature of the effect desired. In general, the dosage of the agent is generally in the range of from about 0.001 to about 50 mg/kg body weight of the subject per day, preferably from about 0.1 to about 10 mg/kg body weight of the subject per day, administered as a single or divided dose. However, some variability in the general dosage range may also be required depending upon the age, weight, and species of the patient, the intended route of administration, and the progress and degree of severity of the disease or condition being treated.

Daily dosages of the agent interacting with a CB1 antagonist required in practicing the method of the present invention will vary depending upon, for example the mode of administration and the severity of the condition to be treated. An indicated daily dose is in the range of from about 1 to about 500 mg, e.g. from 1 to 100 mg of active agent for oral use, conveniently administered once or in divided dosages.

Preferred are combinations, such as combined preparations or pharmaceutical compositions, respectively, comprising a DPP-IV inhibitor preferably vildagliptin or a pharmaceutically accepted salt thereof and as second active agent an active agent selected from the group consisting of Rimonabant, AM-630, AM251, AM281, LY-320135, HU-210, HU-243, O-823, O-2093, SLV 326 or in any case a pharmaceutically accepted salt thereof.

The corresponding active ingredients or a pharmaceutically acceptable salt thereof may also be used in form of a solvate, such as a hydrate or including other solvents, used for crystallization.

The compounds to be combined can be present as pharmaceutically acceptable salts. If these compounds have, for example, at least one basic center, they can form acid addition salts. Corresponding acid addition salts can also be formed having, if desired, an additionally present basic center. The compounds having an acid group (for example COOH) can also form salts with bases.

All of these marketed products may be utilized in as such for combination therapy according to the present invention.

The structure of the active agents identified by generic or tradenames may be taken from the actual edition of the standard compendium “The Merck Index” or from databases, e.g. Patents International (e.g. IMS World Publications). The corresponding content thereof is hereby incorporated by reference. Any person skilled in the art is fully enabled to identify the active agents and, based on these references, likewise enabled to manufacture and test the pharmaceutical indications and properties in standard test models, both in vitro and in vivo.

All the more surprising is the experimental finding that the combined administration of a DPP IV inhibitor or a salt thereof and at least one CB1 antagonist or a salt thereof, results not only in a beneficial, especially a synergistic, therapeutic effect, but also in additional benefits resulting from the combined treatment and further surprising beneficial effects compared to a monotherapy applying only one of the pharmaceutically active compounds used in the combinations disclosed herein. The combination results in an unexpected improvement of the safety profile and therapeutic profile. Additional benefits are e.g. reduction of gastrointestinal diseases an disorders, especially when induced by the CB1 antagonist, reduction of nervous system side effect, especially when induced by the CB1 antagonist. The combination results in an unexpected improvement of the safety profile and therapeutic profile.

It can be shown by established test models and especially those test models described herein that the combination of the DPP-IV inhibitor with at least one CB1 antagonist results in a more effective prevention or preferably treatment of diseases specified in the following. In particular, it can be shown by established test models and especially those test models described herein that the combination of the present invention results in a more effective prevention or preferably treatment of diseases specified hereinafter.

If taken simultaneously, this results not only in a further enhanced beneficial, especially a synergistic, therapeutic effect, but also in additional benefits resulting from the simultaneous treatment such as a surprising prolongation of efficacy, a broader variety of therapeutic treatment and surprising beneficial effects on obesity, appetency disorders or substance abuse disorders, and conditions/disorders that might be treated by DPP-IV inhibition, in particular obesity, diabetes especially type II diabetes, IGT and diseases and conditions associated with diabetes mellitus, IGT, obesity, Parkinson's disease, schizophrenia, Alzheimer's disease, or appetency disorders, for a number of combinations as described herein.

The term “potentiation” shall mean an increase of a corresponding pharmacological activity or therapeutical effect, respectively. Potentiation of one component of the combination according to the present invention by co-administration of another component according to the present invention means that an effect is being achieved that is greater than that achieved with one component alone.

The term “synergistic” shall mean that the drugs, when taken together, produce a total joint effect that is greater than the sum of the effects of each drug when taken alone.

Moreover, for a human patient, especially for elderly people, it is more convenient and easier to remember to take two tablets at the same time, e.g. before a meal, than staggered in time, i.e. according to a more complicated treatment schedule. More preferably, both active ingredients are administered as a fixed combination, i.e. as a single tablet, in all cases described herein. Taking a single tablet is even easier to handle than taking two tablets at the same time. Furthermore, the packaging can be accomplished with less effort.

The person skilled in the pertinent art is fully enabled to select a relevant and standard animal test model to prove the hereinbefore and hereinafter indicated therapeutic indications and beneficial effects.

The pharmaceutical activities as effected by administration of the combination of the active agents used according to the present invention can be demonstrated e.g. by using corresponding pharmacological models known in the pertinent art.

The insulin secretion enhancing properties of the combination according to the present invention may be determined by following the methodology as disclosed, for example, in the publication of T. Ikenoue et al. Biol. Pharm. Bull. 29(4), 354-359 (1997).

The corresponding subject matter of these references is herewith incorporated by reference in this specification.

Accordingly, the combination according to the present invention may be used, e.g., for the prevention, delay of progression or treatment of diseases and disorders that may be inhibited by DPP IV inhibition and/or appetency disorders or nicotinic addiction.

Thus in a further aspect the present invention concerns the use of a combination comprising
i) a DPP IV inhibitor or a pharmaceutically acceptable salt thereof, and
ii) at least one CB1 antagonist, or a pharmaceutically acceptable salt thereof for the manufacture of a medicament for the prevention, delay of progression or treatment of diseases and disorders that may be inhibited by DPP IV inhibition and/or appetency disorders and/or substance abuse disorders.

The invention furthermore relates to a method for the prevention of, delay of progression of, treatment of diseases and disorders that may be inhibited by DPP IV inhibition and/or appetency disorders and/or substance abuse disorders,

comprising administering to a warm-blooded animal, including man, in need thereof a jointly effective amount of a combination of a DPP IV inhibitor or a pharmaceutically acceptable salt thereof with at least one cannabinoid antagonist preferably a CB1 antagonist, or a pharmaceutically acceptable salt thereof;

and at least one additional pharmaceutically acceptable carrier.

The invention furthermore relates to a pharmaceutical composition for the prevention of, delay of progression of, treatment of a disease or condition selected from diseases and disorders that may be inhibited by DPP IV inhibition and/or appetency disorders and/or substance abuse disorders, comprising a combination of a DPP IV inhibitor or a pharmaceutically acceptable salt thereof with at least one therapeutic agent selected from a cannabinoid antagonist preferably a CB1 antagonist, or a pharmaceutically acceptable salt thereof;

and at least one additional pharmaceutically acceptable carrier.

Pharmaceutical composition, methods or uses as described above, wherein the disease or condition is selected from insulin resistance, impaired glucose metabolism, conditions of impaired glucose tolerance, conditions of impaired fasting plasma glucose, diabetes particularly type 2 diabetes mellitus, obesity, diabetic retinopathy, macular degeneration, cataracts, diabetic nephropathy, glomerulosclerosis, diabetic neuropathy, erectile dysfunction, premenstrual syndrome, coronary heart disease, hypertension, angina pectoris, myocardial infarction, stroke, vascular restenosis, skin and connective tissue disorders, foot ulcerations, ulcerative colitis, endothelial dysfunction, impaired vascular compliance, neurodegenerative disorders, cognitive disorders, memory and learning ability problems, appetency disorders and substance abuse disorders or for body fat reduction.

Pharmaceutical composition, methods or uses as described above, wherein, the disease or condition is selected from impaired glucose metabolism, conditions of impaired glucose tolerance, conditions of impaired fasting plasma glucose, diabetes particularly type 2 diabetes mellitus, obesity, diabetic retinopathy, diabetic nephropathy, diabetic neuropathy, foot ulcerations, diseases or conditions associated with diabetes, Parkinson's disease, schizophrenia, Alzheimer's disease, dementia, senile dementia, mild cognitive impairment or Alzheimer type dementia, cognitive deficits associated with schizophrenia, impaired cognitive function associated with Alzheimer's disease, impaired cognitive function associated with Parkinson's disease, appetency disorders or substance abuse disorders, or for body fat reduction.

Pharmaceutical composition, methods or uses as described above, wherein the disease or condition is selected from obesity, diabetes, IGT, type 2 diabetes, Parkinson's disease, schizophrenia, Alzheimer's disease, or appetency disorders.

Pharmaceutical composition, methods or uses as described above, wherein the disease or condition is selected from obesity, diabetes, IGT, type 2 diabetes, Parkinson's disease, schizophrenia, Alzheimer's disease, atherosclerosis, increased cardiovascular morbidity; excess cerebrovascular diseases; increased cardiovascular mortality and sudden death; myocardial infarction, hyperlipidemia, dyslipidemia or appetency disorders.

Pharmaceutical composition, methods or uses as described above, wherein the disease or condition is selected from atherosclerosis, increased cardiovascular morbidity; excess cerebrovascular diseases; increased cardiovascular mortality and sudden death; and myocardial infarction.

Pharmaceutical composition, methods or uses as described above, wherein the disease or condition is selected from obesity, diabetes, IGT, type 2 diabetes, Parkinson's disease, schizophrenia, Alzheimer's disease, atherosclerosis, hyperlipidemia or appetency disorders.

Pharmaceutical composition, methods or uses as described above, wherein the disease or condition is a gastrointestinal disease or disorder.

Pharmaceutical composition, methods or uses as described above, wherein the disease or condition is selected from hyperlipidemia, or conditions associated with hyperlipidemia.

Pharmaceutical composition, methods or uses as described above, for lowering VLDL, LDL and Lp(a) levels in a mammal.

Pharmaceutical composition, methods or uses as described above, for modulating hyperlipidemia, or for modulating conditions associated with hyperlipidemia.

Pharmaceutical composition, methods or uses as described above, wherein the disease or condition is selected from mortality and morbidity after myocardial infarction, adverse cardiac remodeling after myocardial infarction, microvascular complications; increased cardiovascular morbidity; excess cerebrovascular diseases; increased cardiovascular mortality and sudden death; higher incidences and mortality rates of malignant neoplasms.

Pharmaceutical composition, methods or uses as described above, wherein the disease or condition is selected from coronary heart disease, myocardial infarction, diabetic cardiomyopathy, myocardial cell death, coronary artery diseases, peripheral arterial disease, stroke, limb ischemia, vascular restenosis, foot ulcerations, endothelial dysfunction and/or atherosclerosis.

In one further embodiment, the herein described methods, uses and compositions are used for the prevention, or delay of progression of type 2 diabetes.

In one further embodiment, the herein described methods, uses and compositions are used for the prevention, of type 2 diabetes in patients suffering from impaired glucose metabolism, impaired glucose tolerance (IGT) or impaired fasting plasma glucose.

In one further embodiment, the herein described methods, uses and compositions are used to suppress the increased appetite associated with nicotine or tobacco withdrawal.

In one further embodiment, the herein described methods, uses and compositions are used for body fat reduction.

In one further embodiment, the herein described combinations, methods, uses and compositions are used in combination with a further antidiabetic compound which is preferably selected form a glitazone (pioglitazone or rosiglitazone), metformin, or sulfonylureas.

Preferred combinations for the described uses or methods are described herein.

A “disease or condition which may be inhibited by a DPP-IV inhibitor” as defined in this application comprises, but is not limited to insulin resistance, impaired glucose metabolism, conditions of impaired glucose tolerance (IGT), conditions of impaired fasting plasma glucose, diabetes particularly type 2 diabetes mellitus, obesity, diabetic retinopathy, macular degeneration, cataracts, diabetic nephropathy, glomerulosclerosis, diabetic neuropathy, erectile dysfunction, premenstrual syndrome, coronary heart disease, hypertension, angina pectoris, myocardial infarction, stroke, vascular restenosis, skin and connective tissue disorders, foot ulcerations and ulcerative colitis, endothelial dysfunction and impaired vascular compliance, diseases or conditions associated with diabetes, neurodegenerative disorders, cognitive disorders and memory and learning ability problems. The neurodegenerative disorder is selected from Parkinson's disease, schizophrenia, dementia, senile dementia, mild cognitive impairment, Alzheimer related dementia, Huntington's chorea, tardive dyskinesia, hyperkinesias, mania, Morbus Parkinson, steel-Richard syndrome, Down's syndrome, myasthenia gravis, nerve and brain trauma, vascular amyloidosis, cerebral haemorrhage with amyloidosis, brain inflammation, Friedrich's ataxia, acute confusion disorders, acute confusion disorders in which apoptotic necrocytosis plays a part, amyotrophic lateral sclerosis, glaucoma, and Alzheimer's disease. The cognitive disorder is selected from cognitive deficits associated with schizophrenia, age-induced memory impairment, cognitive deficits associated with psychosis, cognitive impairment associated with diabetes, cognitive deficits associated with post-stroke, memory defects associated with hypoxia, cognitive and attention deficits associated with senile dementia, attention-deficit disorders, memory problems associated with mild cognitive impairment, impaired cognitive function associated with dementias, impaired cognitive function associated with Alzheimer's disease, impaired cognitive function associated with Parkinson's disease, impaired cognitive function associated with vascular dementia, cognitive problems associated with brain tumors, Pick's disease, cognitive deficits due to autism, cognitive deficits post electroconvulsive therapy, cognitive deficits associated with traumatic brain injury, amnesic disorders, deliriums, dementias.

The term a “disease or condition which may be inhibited by a DPP-IV inhibitor” does also cover diseases, disorders or conditions related to diabetes, mortality and morbidity after myocardial infarction, adverse cardiac remodeling after myocardial infarction, microvascular complications; increased cardiovascular morbidity; excess cerebrovascular diseases; increased cardiovascular mortality and sudden death; higher incidences and mortality rates of malignant neoplasms.

The term a “disease or condition which may be inhibited by a DPP-IV inhibitor” does also cover gastrointestinal diseases and disorders selected from altered gastrointestinal motility, sensitivity and/or secretion disorder(s) which include, but are not limited to, heartburn, bloating, postoperative ileus, abdominal pain and discomfort, early satiety, epigastric pain, nausea, vomiting, burbulence, regurgitation, intestinal pseudoobstruction, anal incontinence, GERD, IBS, dyspepsia, chronic constipation or diarrhea, diabetic gastropathy, gastroparesis, e.g. diabetic gastroparesis, ulcerative colitis, Crohn's disease, ulcers and the visceral pain associated therewith.

The term a “disease or condition which may be inhibited by a DPP-IV inhibitor” does also cover hyperlipidemia and/or conditions associated with hyperlipidemia.

Hyperlipidemia is an important precipitating factor for the premature development of atherosclerosis and increased rate of cardiovascular and peripheral vascular diseases. Hyperlipidemia is a condition generally characterized by an abnormal increase in serum lipids in the bloodstream and is an important risk factor in developing atherosclerosis and heart disease. For a review of disorders of lipid metabolism, see, e.g., Wilson, et al., Ed., Disorders of Lipid Metabolism, Chapter 23, Textbook of Endocrinology, 9^{th} Edition, W.B. Sanders Company, Philadelphia, Pa. (1998); this reference and all references cited therein are herein incorporated by reference. Serum lipoproteins are the carriers for lipids in the circulation and include chylomicrons, very low-density lipoproteins (VLDL), intermediate density lipoproteins (IDL), low density lipoproteins (LDL) and high density lipoproteins (HDL) and lipoprotein a (Lp(a)). Hyperlipidemia is usually classified as primary or secondary hyperlipidemia. Primary hyperlipidemia is generally caused by genetic defects, while secondary hyperlipidemia is generally caused by other factors, such as various disease states, drugs and dietary factors. Alternatively, hyperlipidemia can result from both a combination of primary and secondary causes of hyperlipidemia. Elevated cholesterol levels are associated with a number of disease states, including coronary artery disease, angina pectoris, carotid artery disease, strokes, cerebral arteriosclerosis, and xanthoma.

There are several forms of circulating blood cholesterol which occur naturally in mammals. Some forms are considered “bad” cholesterol, while other forms are considered “good” cholesterol and are essential for good health. The good form of cholesterol has been established to be HDL. LDL is a “bad” cholesterol. Another form of LDL cholesterol, the primary bad form, is Lp(a) which is a modified form of LDL. Elevated levels of Lp(a) are believed to be detrimental and associated with a higher risk for coronary heart disease (CHD) (see Assman et al., Am. J. Card., Vol. 77, pp. 1179-1184 (1996); and Bostom et al., JAMA, Vol. 276, No. 7, pp. 544-548 (1996)).

The term “hyperlipidemia” refers to the presence of an abnormally elevated level of lipids in the blood. Hyperlipidemia can appear in at least three forms: (1) hypercholesterolemia, i.e., an elevated cholesterol level; (2) hypertriglyceridemia, i.e., an elevated triglyceride level; and (3) combined hyperlipidemia, i.e., a combination of hypercholesterolemia and hypertriglyceridemia. This term also refers to elevated levels of one or more lipoproteins, e.g., elevated levels of Lp(a), LDL and/or VLDL.

The term “elevated levels of Lp(a)” as used herein shall mean levels of Lp(a) which subjects the patient to the risk of vascular, particularly cardiovascular diseases, mediated by Lp(a), including but not limited to CHD, ischemic stroke, restenosis after angioplasty, peripheral vascular disease, intermittent claudication, myocardial infarction (e.g. reduction in necrosis), dyslipidemia and post-prandial lipemia.

The term “cholesterol” refers to a steroid alcohol that is an essential component of cell membranes and myelin sheaths and, as used herein, incorporates its common usage. Cholesterol also serves as a precursor for steroid hormones and bile acids.

The term “triglyceride(s)” (TGs), as used herein, incorporates its common usage. TGs consist of three fatty acid molecules esterified to a glycerol molecule and serve to store fatty acids which are used by muscle cells for energy production or are taken up and stored in adipose tissue.

Because cholesterol and TGs are water insoluble, they must be packaged in special molecular complexes known as “lipoproteins” in order to be transported in the plasma. Lipoproteins can accumulate in the plasma due to overproduction and/or deficient removal. There are at least five distinct lipoproteins differing in size, composition, density and function. In the cells of the small of the intestine, dietary lipids are packaged into large lipoprotein complexes called “chylomicrons”, which have a high TG and low cholesterol content. In the liver, TG and cholesterol esters are packaged and released into plasma as TG-rich lipoprotein called VLDL, whose primary function is the endogenous transport of TGs made in the liver or released by adipose tissue. Through enzymatic action, VLDL can be either reduced and taken up by the liver, or transformed into IDL. IDL, is in turn, either taken up by the liver, or is further modified to form the LDL. LDL is either taken up and broken down by the liver, or is taken up by extrahepatic tissue. HDL helps remove cholesterol from peripheral tissues in a process called reverse cholesterol transport.

Exemplary primary hyperlipidemia include, but are not limited to, the following:

1) Familial hyperchylomicronemia, a rare genetic disorder which causes a deficiency in an enzyme, LP lipase, that breaks down fat molecules. The LP lipase deficiency can cause the accumulation of large quantities of fat or lipoproteins in the blood;

2) Familial hypercholesterolemia, a relatively common genetic disorder caused where the underlying defect is a series of mutations in the LDL receptor gene that result in malfunctioning LDL receptors and/or absence of the LDL receptors. This brings about ineffective clearance of LDL by the LDL receptors resulting in elevated LDL and total cholesterol levels in the plasma;

3) Familial combined hyperlipidemia, also known as multiple lipoprotein-type hyperlipidemia; an inherited disorder where patients and their affected first-degree relatives can at various times manifest high cholesterol and high triglycerides. Levels of HDL cholesterol are often moderately decreased;

4) Familial defective apolipoprotein B-100 is a relatively common autosomal dominant genetic abnormality. The defect is caused by a single nucleotide mutation that produces a substitution of glutamine for arginine which can cause reduced affinity of LDL particles for the LDL receptor. Consequently, this can cause high plasma LDL and total cholesterol levels;

5) Familial dysbetaliproteinemia, also referred to as Type III hyperlipoproteinemia, is an uncommon inherited disorder resulting in moderate to severe elevations of serum TG and cholesterol levels with abnormal apolipoprotein E function. HDL levels are usually normal; and

6) Familial hypertriglyceridemia, is a common inherited disorder in which the concentration of plasma VLDL is elevated. This can cause mild to moderately elevated triglyceride levels (and usually not cholesterol levels) and can often be associated with low plasma HDL levels. Risk factors in exemplary secondary hyperlipidemia include, but are not limited to, the following: (1) disease risk factors, such as a history of Type 1 diabetes, Type 2 diabetes, Cushing's syndrome, hypothyroidism, cholestasis and certain types of renal failure; (2) drug risk factors, which include, birth control pills; hormones, such as estrogen and corticosteroids; certain diuretics; and various β-blockers; (3) dietary risk factors include dietary fat intake per total calories greater than 40%; saturated fat intake per total calories greater than 10%; cholesterol intake greater than 300 mg per day; habitual and excessive alcohol use; bulimia, anorexia nervosa, and obesity.

The term conditions associated with hyperlipidemia are selected from the group consisting of atherosclerosis, angina pectoris, carotid artery disease, cerebral arteriosclerosis, xanthoma, CHD, ischemic stroke, restenosis after angioplasty, peripheral vascular disease, intermittent claudication, reduction in necrosis after myocardial infarction, dyslipidemia, post-prandial lipemia.

In a first embodiment, a “disease or condition which may be inhibited by a DPP-IV inhibitor” is selected from impaired glucose metabolism, conditions of impaired glucose tolerance, conditions of impaired fasting plasma glucose, diabetes particularly type 2 diabetes mellitus, obesity, diabetic retinopathy, diabetic nephropathy, diabetic neuropathy, foot ulcerations, diseases or conditions associated with diabetes, Parkinson's disease, schizophrenia, Alzheimer's disease, dementia, senile dementia, mild cognitive impairment or Alzheimer type dementia, cognitive deficits associated with schizophrenia, impaired cognitive function associated with Alzheimer's disease, impaired cognitive function associated with Parkinson's disease.

In a second embodiment a “disease or condition which may be inhibited by a DPP-IV inhibitor” is selected from hyperlipidemia, conditions associated with hyperlipidemia and/or high blood level of VLDL, LDL and Lp(a) levels in a mammal.

In a second embodiment a “disease or condition which may be inhibited by a DPP-IV inhibitor” is selected from mortality and morbidity after myocardial infarction, adverse cardiac remodeling after myocardial infarction, microvascular complications; increased cardiovascular morbidity; excess cerebrovascular diseases; increased cardiovascular mortality and sudden death; higher incidences and mortality rates of malignant neoplasms.

In a third embodiment a “disease or condition which may be inhibited by a DPP-IV inhibitor” is selected from coronary heart disease, myocardial infarction, diabetic cardiomyopathy, myocardial cell death, coronary artery diseases, peripheral arterial disease, stroke, limb ischemia, vascular restenosis, foot ulcerations, endothelial dysfunction and/or atherosclerosis.

The term “curative” as used herein means efficacy in treating ongoing diseases, disorder or conditions.

The term “prophylactic” means the prevention of the onset or recurrence of diseases, disorders or conditions to be treated.

The term “delay of progression” as used herein means administration of the combination to patients being in a pre-stage or in an early phase of the disease to be treated, in which patients for example a pre-form of the corresponding disease is diagnosed or which patients are in a condition, e.g. during a medical treatment or a condition resulting from an accident, under which it is likely that a corresponding disease will develop.

In the present description and in the claims, “appetency disorders” are understood as meaning disorders associated with a substance and especially abuse of a substance and/or dependency on a substance, disorders of food behaviors, especially those liable to cause excess weight, irrespective of its origin, for example: bulimia, appetency for sugars, non-insulin-dependent diabetes. Appetizing substances are therefore understood as meaning substances to be taken into the body and for which an appetite or craving for such consumption by any route of entry. Appetizing substances include, but are not limited to, foods, and their appetizing ingredients such as sugars, carbohydrates, or fats, as well as drinking alcohol or drugs of abuse or excess consumption. An “appetite” may be directed toward such substances as foods, sugars, carbohydrates, fats, as well as ethanol or drugs of abuse or addiction or excess consumption (e.g., tobacco, CNS depressants, CNS stimulants). In one embodiment, the disorder is increased appetite associated with nicotine or tobacco withdrawal. Thus the term “appetency disorders” covers also treatment for reducing body weight or reducing body fat or reducing appetite for food or reducing food intake or consumption or causing hypophagia in mammals (e.g., humans, cats or dogs). The term “appetency disorders” can also cover a treatment to reduce appetite for food.

As used herein, the term “substance abuse disorders” includes substance dependence or abuse with or without physiological dependence. The substances associated with these disorders are: alcohol, amphetamines (or amphetamine-like substances), caffeine, cannabis, cocaine, hallucinogens, inhalants, marijuana, nicotine, opioids, phencyclidine (or phencyclidine-like compounds), sedative-hypnotics or benzodiazepines, and other (or unknown) substances and combinations of all of the above.

In particular, the term “substance abuse disorders” includes drug withdrawal disorders such as alcohol withdrawal with or without perceptual disturbances; alcohol withdrawal delirium; amphetamine withdrawal; cocaine withdrawal; nicotine withdrawal; opioid withdrawal; sedative, hypnotic or; anxiolytic withdrawal with or without perceptual disturbances; sedative, hypnotic or anxiolytic withdrawal delirium; and withdrawal symptoms due to other substances. Thus the term “substance abuse disorders” covers also a treatment to suppress the increased appetite associated with nicotine or tobacco withdrawal, or the treatment of addiction to psychoactive substances such as narcotics, CNS stimulants, CNS depressants, and anxyiolytics.

It will be appreciated that reference to treatment of nicotine withdrawal includes the treatment of symptoms associated with smoking cessation.

Other “substance abuse disorders” include substance-induced anxiety disorder with onset during withdrawal; substance-induced mood disorder with onset during withdrawal; and substance-induced sleep disorder with onset during withdrawal.

The term “body fat reduction” means loss of a portion of body fat.

The formula for Body Mass Index (BMI) is [Weight in pounds÷Height in inches÷Height in inches]×703. BMI cutpoints for human adults are one fixed number, regardless of age or sex, using the following guidelines: Overweight human adults individuals have a BMI of 25.0 to 29.9. Obese human adults have a BMI of 30.0 or more. Underweight adults have a BMI less of than 18.5. A normal body weight range for an adult is defined as a BMI between 18.5 and 25. BMI cutpoints for children under 16 are defined according to percentiles: Overweight is defined as a BMI for age greater than>85th percentile and obesity is defined as a BMI-for-age>95th percentile. Underweight is a BMI-for-age<5th percentile. A normal body weight range for a child is defined as a BMI above the 5th percentile and below the 85 percentile.

The term “combined pharmaceutical preparation” as that term is used herein means that the active ingredients, e.g. rimonabant and a DPP-IV inhibitor preferably LAF237, are both administered to a patient as separate entities either simultaneously, concurrently or sequentially with no specific time limits, wherein such administration provides therapeutically effective levels of the two compounds in the body, preferably at the same time. As an example, a non-fixed combination would be two capsules each containing one active ingredient where the purpose is to have the patient achieve treatment with both active ingredients together in the body.

The term “fatty acid oxidation” relates to the conversion of fatty acids (e.g., oleate) into ketone bodies.

The term “modulate” means to induce any change including increasing or decreasing. (e.g., a modulator of fatty acid oxidation increases or decreases the rate of fatty oxidation, a modulator of a receptor includes both agonists and antagonists of the receptor).

The term “muscle cells” refers to cells derived from the predominant cells of muscle tissue. Muscle cells may be freshly isolated from muscle tissue or established cell lines.

The term “weight loss” refers to loss of a portion of total body weight.

The term “treat” or “treatment” encompasses the complete range of therapeutically positive effects associated with pharmaceutical medication including reduction of, alleviation of and relief from the symptoms or illness which affect the organism.

Preferably, the jointly therapeutically effective amounts of the active agents according to the combination of the present invention can be administered simultaneously or sequentially in any order, e.g. separately (combined pharmaceutical preparation) or in a fixed combination.

Under certain circumstances, drugs with different mechanisms of action may be combined. However, just considering any combination of drugs having different modes of action but acting in the similar field does not necessarily lead to combinations with advantageous effects.

All the more surprising is the experimental finding that the combined administration of a DPP-IV inhibitor according to the present invention, or, in each case, a pharmaceutically acceptable form thereof, results not only in a beneficial, especially a potentiating or a synergistic, therapeutic effect. Independent thereof, additional benefits resulting from combined treatment can be achieved such as a surprising prolongation of efficacy, a broader variety of therapeutic treatment and surprising beneficial effects on diseases and conditions associated with diabetes (e.g. less appetite, less gain of weight or less cardiovascular side effects).

The diseases, disorders or conditions related to diabetes, particularly type 2 diabetes mellitus, includes but are not limited to diabetic nephropathy, diabetic retinopathy and diabetic neuropathy, macular degeneration, coronary heart disease, myocardial infarction, diabetic cardiomyopathy, myocardial cell death, coronary artery diseases, peripheral arterial disease, stroke, limb ischemia, vascular restenosis, foot ulcerations, endothelial dysfunction and/or atherosclerosis.

Further benefits are that lower doses of the individual drugs to be combined according to the present invention can be used to reduce the dosage, for example, that the dosages need not only often be smaller but are also applied less frequently, or can be used in order to diminish the incidence of side effects. This is in accordance with the desires and requirements of the patients to be treated.

For example, it has turned out that the combination according to the present invention provides benefit especially in the treatment of diabetic patients, e.g. reducing the risk of negative cardiovascular events, reducing risk of side effects, controlling increase of weight (in diabetic patients) or in patients suffering from an altered gastrointestinal motility, sensitivity and/or secretion disorder(s).

In view of reduced dose of the DPP-IV inhibitor or CB1 antagonist, used according to the present invention, there is a considerable safety profile of the combination making it suitable for first line therapy.

The pharmaceutical composition according to the present invention as described herein before and hereinafter may be used for simultaneous use or sequential use in any order, for separate use or as a fixed combination.

Method or use as described above, wherein the DPP-IV inhibitor and the CB1 antagonist are administered in the form of a combination of the present invention such as a fixed combination or combined preparation or kit of part.

“kit-of-parts”, combination, method or use as described herein, wherein the DPP-IV inhibitor is vildagliptin or and wherein the CB1 antagonist is preferably selected from the group consisting of Rimonabant, AM-630, AM251, AM281, LY-320135, HU-210, HU-243, O-823, O-2093, SLV 326, or in each case, a pharmaceutically acceptable salt thereof.

“kit-of-parts”, combination, method or use as described above, wherein the DPP-IV inhibitor is vildagliptin and wherein the CB1 antagonist is Rimonabant, or in each case, a pharmaceutically acceptable salt thereof.

According the invention, when the DPP-IV inhibitors, and the CB1 antagonist are administered together, such administration can be sequential in time or simultaneous with, the simultaneous method being generally preferred. For sequential administration, the DPP-IV inhibitor, and the CB1 antagonist can be administered in any order. It is generally preferred that such administration be oral. It is especially preferred that the administration be oral and simultaneous. However, if the subject being treated is unable to swallow, or oral absorption is otherwise impaired or undesirable, parenteral or transdermal administration will be appropriate. When the DPP-IV inhibitor, and the CB1 antagonist are administered sequentially, the administration of each can be by the same method or by different methods.

A further aspect of the present invention is a kit for the prevention of, delay of progression of, treatment of a disease or condition according to the present invention comprising

(a) an amount of a DPP IV inhibitor or a pharmaceutically acceptable salt thereof in a first unit dosage form;

(b) an amount of at least one CB1 antagonist or, in each case, where appropriate, a pharmaceutically acceptable salt thereof in a second etc. unit dosage form; and

(c) a container for containing said first, second etc. unit forms.

In a variation thereof, the present invention likewise relates to a “kit-of-parts”, for example, in the sense that the components to be combined according to the present invention can be dosed independently or by use of different fixed combinations with distinguished amounts of the components, i.e. simultaneously or at different time points. The parts of the kit of parts can then e.g. be administered simultaneously or chronologically staggered, that is at different time points and with equal or different time intervals for any part of the kit of parts. Preferably, the time intervals are chosen such that the effect on the treated disease or condition in the combined use of the parts is larger than the effect that would be obtained by use of only any one of the components.

The present invention thus also relates to a kit of parts comprising

(a) an amount of a DPP IV inhibitor or a pharmaceutically acceptable salt thereof in a first unit dosage form;

(b) an amount of at least one CB1 antagonist or, in each case, where appropriate, a pharmaceutically acceptable salt thereof,

in the form of two or three or more separate units of the components (a) to (b), especially for the prevention of, delay of progression of, treatment of a disease or condition according to the present invention.

The invention furthermore relates to a commercial package comprising the combination according to the present invention together with instructions for simultaneous, separate or sequential use.

In a preferred embodiment, the (commercial) product is a commercial package comprising as active ingredients the combination according to the present invention (in the form of two or three or more separate units of the components (a) or (b)), together with instructions for its simultaneous, separate or sequential use, or any combination thereof, in the delay of progression or treatment of the diseases as mentioned herein.

All the preferences mentioned herein apply to the combination, composition, use, method of treatment, “kit of parts” and commercial package of the invention.

In an additional embodiment, the present invention concerns;
1. the use of a DPP IV inhibitor or a pharmaceutically acceptable salt thereof, for the manufacture of a medicament for the prevention, delay of progression or treatment of appetency disorders, substance abuse disorders or for body fat reduction.
2. a method for the prevention of, delay of progression of, treatment of appetency disorders and/or substance abuse disorders and/or for body fat reduction, comprising administering to a warm-blooded animal, including man, in need thereof an effective amount of a DPP IV inhibitor or a pharmaceutically acceptable salt thereof and at least one additional pharmaceutically acceptable carrier.
3. a pharmaceutical composition for the prevention of, delay of progression of, treatment of appetency disorders and/or substance abuse disorders, comprising a DPP IV inhibitor or a pharmaceutically acceptable salt thereof;
   and at least one additional pharmaceutically acceptable carrier.

These pharmaceutical preparations are for enteral, such as oral, and also rectal or parenteral, administration to homeotherms, with the preparations comprising the pharmacological active compound either alone or together with customary pharmaceutical auxiliary substances. For example, the pharmaceutical preparations consist of from about 0.1% to 90%, preferably of from about 1% to about 80%, of the active compound. Pharmaceutical preparations for enteral or parenteral, and also for ocular, administration are, for example, in unit dose forms, such as coated tablets, tablets, capsules or suppositories and also ampoules. These are prepared in a manner that is known per se, for example using conventional mixing, granulation, coating, solubulizing or lyophilizing processes. Thus, pharmaceutical preparations for oral use can be obtained by combining the active compound(s) with solid excipients, if desired granulating a mixture which has been obtained, and, if required or necessary, processing the mixture or granulate into tablets or coated tablet cores after having added suitable auxiliary substances.

The dosage of the active compound can depend on a variety of factors, such as mode of administration, homeothermic species, age and/or individual condition.

Preferred dosages for the active ingredients of the pharmaceutical combination according to the present invention are therapeutically effective dosages, especially those which are commercially available.

Normally, in the case of oral administration, an approximate daily dose of from about 1 mg to about 360 mg is to be estimated e.g. for a patient of approximately 75 kg in weight. The dosage of the active compound can depend on a variety of factors, such as mode of administration, homeothermic species, age and/or individual condition.

The pharmaceutical preparation will be supplied in the form of suitable dosage unit form, for example, a capsule or tablet, and comprising an amount, being together with the further component(s) jointly effective, e.g. 100 mg or 50 mg of vildagliptin.

The pharmaceutical composition according to the present invention as described hereinbefore may be used for simultaneous use or sequential use in any order, for separate use or as a fixed combination.

Thus according to a further embodiment, a DPP-IV inhibitor, is administered with a CB1 antagonist, preferably in the form of a fixed pharmaceutical composition comprising a pharmaceutically acceptable carrier, vehicle or diluent. Accordingly, a DPP-IV inhibitor of this invention, can be administered with a CB1 antagonist as a fixed combination, in any conventional oral, parenteral or transdermal dosage form.

The doses of DPP-IV inhibitor of formula (I) to be administered to warm-blooded animals, for example human beings, of, for example, approximately 70 kg body weight, especially the doses effective in the inhibition of the DPP-IV enzyme, are from approximately 3 mg to approximately 3 g, preferably from approximately 10 mg to approximately 1 g, for example approximately from 20 mg to 200 mg, per person per day, divided preferably into 1 to 4 single doses which may, for example, be of the same size. Usually, children receive about half of the adult dose. The dose necessary for each individual can be monitored, for example by measuring the serum concentration of the active ingredient, and adjusted to an optimum level. Single doses comprise, for example, 10, 40, 50, 100 or 150 mg per adult patient.

The dosage of vildagliptin is preferably between 10 and 150 mg daily, most preferably between 25 and 150 mg, 25 and 100 mg or 25 and 50 mg or 50-100 mg daily. Preferred examples of daily oral dosage are 25, 30, 35, 45, 50, 55, 60, 80, 100 or 150 mg. The application of the active ingredient may occur up to three times a day, preferably one or two times a day.

The preferred herein mentioned CB1 antagonists will be supplied in the form of suitable dosage unit form, for example, a capsule or tablet, and comprising a therapeutically effective amount, e.g. from about 2 to about 200 mg, as already described herein and in the prior art. The application of the active ingredient may occur up to three times a day, preferably one or two times a day. The same preferred dosage are selected for the fixed combinations.

Combination, composition, method or use, as described herein, wherein the CB1 antagonist or a pharmaceutically acceptable salt thereof, is administered in an amount between 5 and 80 mg or between 5 and 20 mg daily.

Daily rimonabant dosages required in practicing the method of the present invention will vary depending upon, for example the mode of administration and the severity of the condition to be treated. An indicated daily dose is in the range of from about 1 to about 100 mg, e.g. from 5 to 50 mg or from 5 to 20 mg, of active agent for oral use, conveniently administered once or in divided dosages.

Corresponding doses may be taken, for example, in the morning, at mid-day or in the evening.

In a preferred aspect, the invention concerns a “kit-of-parts”, combination, use or a method as described herein, comprising or wherein the daily administration is;
i) between 25 and 150 mg or between 50 and 100 mg of vildagliptin, and
ii) between 5 and 50 mg or between 5 and 20 mg of rimonabant,
   or in any case, a pharmaceutically acceptable salt thereof.

In a preferred aspect, the invention concerns a “kit-of-parts”, combination or use or a method as described herein, comprising or wherein the daily administration is;
i) 50, 100 or 150 mg of vildagliptin, and
ii) 5, 10 or 20 mg of rimonabant,
   or in any case, a pharmaceutically acceptable salt thereof.

Preferably, in case of free combinations, preferred are those dosages for launched products that have been approved and that have been marketed.

Especially preferred are low dose combinations.

To further illustrate the invention, but not by way of limitation, the following examples are provided.

A) Bioassay Methods for Assessing the Effects of Compounds and Combination Therapies on Appetite(s), Body Fat Reduction, Body Weight, and Lipid Metabolism.

The dose(s) administered to the animal are sufficient to determine if the compounds or combination therapy has a desired effect, for example, an appetite, body weight, body fat, and/or fatty acid oxidation over time. Such dose(s) can be determined according to the efficacy of the particular candidate compound(s) employed and the condition of the animal, as well as the body weight or surface area of the animal. The size of the dose(s) also will be determined by the existence, nature, and extent of any adverse side effects that accompany the administration of a candidate compound or combination; the LD50 of the candidate compound or combination; and the side-effects of the candidate compound or combination at various concentrations. Depending upon the compound or combination and the above factors, for instance, the initial test dosage(s) may range, for example, from 0.1-50 mg per kg, preferably 1-25 mg per kg, most preferably 1-20 mg per kg body weight for each of the compound or combination. The determination of dose response relationships is well known to one of ordinary skill in the art.

Test animals subjects can be, for example, obese or normal mammals (e.g., humans, primates, guinea pigs, rats, mice, or rabbits). Suitable rats include, but are not limited to, Zucker rats. Suitable mice include, but are not limited to, for example, ALS/LtJ, C3. SW-H-2b/SnJ, (NON/LtJ×NZO/HIJ)F1, NZO/H1J, ALR/LtJ, NON/LtJ, KK.Cg-AALR/LtJ, NON/LtJ, KK.Cg-Ay/J, B6.HRS(BKS)-Cpefat+, B6.129P2-Gcktm/Efr, B6.V-Lepob, BKS.Cg-m+/+Leprdb, and C57BL/6J with Diet Induced Obesity.

A. Assessing Effects on Appetite, Including Food Consumption.

The effect of the candidate compounds and combinations i.e. DPP-IV inhibitors (vildagliptin) and CB1 antagonists (rimonabant) or combination of such compounds on an appetite for appetizing substance (e.g., sugar, ethanol, a psychoactive substance such as nicotine, narcotics, opiates, CNS stimulants or depressants, anxyiolytic) can be assessed, for instance, by monitoring the consumption of the substance by test subjects (e.g., measuring the amount (e.g., by volume or weight) Consumed or used or not consumed and not used, use of consumption diaries) Or tissue levels (e.g., blood, plasma) Or excretion levels (e.g., urine, feces levels) of the appetitive substance or its metabolites or by monitoring behaviors seeking the appetitive substance. The effect of the compounds and combinations on appetite can also be assessed by subjective means including questionnaires as to appetite or cravings levels by human subjects. The techniques for these assessments are well known to those of ordinary skill in the art. The studies may be acute, subacute, chronic, or subchronic with respect to the duration of administration and or follow-up of the effects of the administration. See also U.S. Pat. No. 6,344,474.

The effect of the candidate compounds and combinations i.e. DPP-IV inhibitors (vildagliptin) and CB1 antagonists (rimonabant) or combination of such compounds on the appetite for food or in inducing hypophagia or reduced food intake can be directly assessed, for instance, by monitoring the food consumption of the test subjects (e. g., measuring the amount eaten or not eaten by a subject in terms of food weight or caloric content). The effect on food consumption can be indirectly measured by monitoring body weight. The effect of the compounds on appetite can also be assessed by food consumption diaries, or subjective means including questionnaires as to appetite or food cravings levels by human subjects. The techniques for these assessments are well known to those of ordinary skill in the art. The studies may be acute, subacute, chronic, or subchronic with respect to the duration of administration and or follow-up of the effects of the administration.

B) Assessing Effects on Body Fat Reduction.

Effects on body fat can be identified in vivo using animal bioassay techniques well known to those of ordinary skill in the art. Body fat reduction is typically determined by direct measurements of the change in body fat or by loss of body weight. Body fat and/or body weight of the animals is determined before, during, and after the administration of the candidate compounds or combinations. Test compounds (DPP-IV inhibitors (vildagliptin) and CB1 antagonists (rimonabant)) or combinations thereof and appropriate vehicle or caloric controls can be administered by any of a number of routes (e.g., the oral route, a parenteral route) to experimental subjects and the weight of the subjects can be monitored over the course of therapy. The experimental subjects can be humans as well as surrogate test animals (e. g., rats, mice).

Changes in body fat are measured by any means known in the art such as, for example, fat fold measurements with calipers, bioelectrical impedance, hydrostatic weighing, or dual x-ray absorbiometry. Preferably animals demonstrate at least 2%, 5%, 8%, or 10% loss of body fat. Changes in body weight can be measured by any means known in the art such as, for example, on a portable scale, on a digital scale, on a balance scale, on a floor scale, or a table scale. Preferably animals demonstrate at least 2%, 5%, 10%, or 15% loss of body weight. Body weight reduction is measured before administration of the candidate compound or combination and at regular intervals during and after treatment. Preferably, body weight is measured every 5 days, more preferably every 4 days, even more preferably every 3 days, yet more preferably every 2 days, most preferably every day.

For instance, the effect of the candidate compounds and combinations on total body fat can be determined by taking direct measurements of the rat's body fat using skin fold calipers. Skin on the subjects' backs, abdomen, chest, front and rear legs can be pinched with calipers to obtain measurements before administration of the test compound and at daily or longer intervals (e.g., every 48 hours) during and after administration of candidate compounds and combinations. Differences in measurements in one or more of the “pinched” sites reflect the change in the rat's total body fat. The animal may selected from any test species, including but not limited to, mammals, the mouse, a rat, a guinea pig, or a rabbit. The animal may also be an ob/ob mouse, a db/db mouse, or a Zucker rat or other animal model for a weight-associated disease. Clinical studies in humans may also be conducted. In humans, body density measurements or estimates of percent body fat may also be used to assess body fat reduction.

C) Assessing Effects on Lipid Metabolism.

The candidate compounds and combinations i.e. DPP-IV inhibitors (vildagliptin) and CB1 antagonists (rimonabant) or combinations of such compounds can also be assayed for their effect on fatty acid metabolism. The effect of the candidate compounds and combinations on fatty acid metabolism can be measured by measurements of fatty acid oxidation in primary cultures of liver cells as taught for instance in U.S. patent application Ser. No.10/112,509 filed on Mar. 27, 2002 and assigned to the same assignee as the present application and incorporated by reference.

Changes in fatty acid metabolism can be measured, for instance, by looking at fatty acid oxidation in cells from major fat burning tissues such as, for example, liver (Beynen, et al., Diabetes, 28:828 (1979)), muscle (Chiasson Lab. Anat. of Rat (1980)), heart (Flink, et al., J. Biol. Chem., 267: 9917 (1992)), and adipocytes (Rodbell, J. Biol. Chem., 239: 375 (1964)), Cells may be from primary cultures or from cell lines. Cells may be prepared for primary cultures by any means known in the art including, for example, enzymatic digestion and dissection. Suitable cell lines are known to those in the art. Suitable hepatocyte lines are, for example, Fao, MH1C1, H-4-II-E, H4TG, H4-II-E-C3, McA-RH7777, McA-RH8994, N1-S1 Fudr, N1-S1, ARL-6, Hepa 1-6, Hepa-1c1c7, BpRc1, tao BpRc1, NCTC clone 1469, PLC/PRF/5, Hep 3B2.1-7 [Hep 3B], Hep G2 [HepG2], SK-HEP-1, WCH-17. Suitable skeletal muscle cell lines are, for example, L6, L8, C8, NOR-10, BLO-11, BC3H1, G-7, G-8, C2C12, P19, Sol8, SJRH30 [RMS 13], QM7. Suitable cardiac cell lines are, for example, H9c2(2-1), P19, CCD-32Lu, CCD-32Sk, Girardi, FBHE. Suitable adipocyte lines are, for example, NCTC clone 929 [derivative of Strain L; L-929; L cell], NCTC 2071, L-M, L-M(TK-) [LMTK-; LM(tk-)], A9 (APRT and HPRT negative derivative of Strain L), NCTC clone 2472, NCTC clone 2555, 3T3-L1, J26, J27-neo, J27-B7, MTKP 97-12 pMp97B [TKMp97-12], L-NGC-5HT2, Ltk-11, L-alpha-1b, L-alpha-2A, L-alpha-2C, B82.

The rate of fatty acid oxidation may be measured by 14C-oleate oxidation to ketone bodies (Guzmán and Geelen Biochem. J. 287:487 (1982)) and/or 14C-oleate oxidation to CO₂ (Fruebis, PNAS, 98:2005 (2001); Blazquez, et al., J. Neurochem, 71: 1597 (1998) ). Lypolysis may be measured by fatty acid or glycerol release by using appropriate labeled precursors or spectrophotometric assays (Serradeil-Le Gal, FEBS Lett, 475: 150 (2000)). For analysis of 14C-oleate oxidation to ketone bodies, freshly isolated cells or cultured cell lines can be incubated with 14C-oleic acid for an appropriate time, such as, for example, 30, 60, 90, 120, or 180 minutes. The amount of 14C radioactivity in the incubation medium can be measured to determine their rate of oleate oxidation. Oleate oxidation can be expressed as nmol oleate produced in x minutes per g cells. For analysis of lypolysis/glycerol release, freshly isolated cells or cultured cells lines can be washed then incubated for an appropriate time. The amount of glycerol released into the incubation media can provide an index for lypolysis.

D) Cannabinoid Receptor Activity Screening.

A variety of means may be used to screen cannabinoid CB1 receptor activity in order to identify the compounds according to the invention. A variety of such methods are taught in U.S. Pat. No. 5,747,524 and U.S. Pat. No. 6,017,919.

E) Bioassay Methods for Assessing the Effects of Compounds, and Combination Therapies on Alzheimer's Disease, Parkinson's Disease, Cognitive Disorders, Memory and Learning Ability Problems.

The pharmacological activity of the compounds i.e. DPP-IV inhibitors (vildagliptin) and CB1 antagonists (rimonabant) and combinations of such compounds according to the invention in improving cognitive function may, for example, be assessed using tests known to a person skilled in the art such as standardized psychometric tests (e.g. Wechsler Memory Scale, the Wechsler Adult Intelligence Scale, Raven's Standard Progressive Matrices, Schaie-Thurstone Adult Mental Abilities Test), neuropsychological tests (e.g. Luria-Nebraska), metacognitive self-evaluations (e.g. Metamemory Questionnaire), visual-spatial screening tests (e.g. Poppelreuter's Figures, Clock Recognition, Honeycomb Drawing and Cancellation), cognitive screening tests (e.g. Folstein's Mini Mental State Test) and reaction time tests. Such standardized tests as listed above are described in Ruoppila, I. and Suutama, T. (1997) Scand. J. Soc. Med. Suppl. 53, 44-65 and serve as examples, said reference is incorporated in its entirety herein. The term “cognitive function” includes the functions assessed by any such test.

A clinical protocol to show the positive effect of a the combination of the invention for treating the Alzheimer's disease development is described in the patent application WO 2004/082706 on pages 31-37, which is incorporated herein by reference.

The examples 2-4 described in the patent application WO2005009349 describe a further protocols to assess the activity of the compounds and combinations of the present invention to treat or prevent, X syndrome, Alzheimer's disease and Parkinson's' disease which is incorporated herein by reference.

Other standard tests for cognitive performance e.g. the Alzheimer's Disease Assessment Scale (ADAS-cog) are described by Doraiswamy (Neurology. 1997 June;48(6):1511-7) and in the patents US20040024043 and U.S. Pat. No. 6369046. The ADAS-cog is a multi-item instrument for measuring cognitive performance which include elements of memory, orientation, retention, reasoning, language and praxis. US20040024043 describes also an in vivo test model in rodents in example 5 and a clinical Study Design in example 9. Another clinical Study Design is described by U.S. Pat. No. 6369046 (example 1) which are incorporated herein by reference.

A further useful in vivo protocol which can be used to show that DPP-IV inhibitors can improving cognitive function is described in the European Patent No.1310258 (examples 5-8), which is incorporated herein by reference.

### EXAMPLE 1

The effects of the herein described combinations i.e. comprising a DPP-IV inhibitors such as vildagliptin and CB1 antagonists such as rimonabant in a model of Parkinson disease are investigated in mice. Male C57/BL6 mice are injected once daily for 7 days with MPTP (30 mg/kg, i.p.). Vildagliptin and/or rimonabant are administered once or twice daily for 14 days. On day 28, striata are removed, homogenized in perchloric acid, and centrifuged. The supernatant is removed and analyzed for dopamine and other monoamines such as serotonin by reverse-phase HPLC and electrochemical detection. Anti-Parkinson activity of the combination Vildagliptin and rimonabant is assessed in comparison to the mono-therapy with either Vildagliptin or rimonabant alone.

### EXAMPLE 2

### Treating, Preventing or Delaying Cognitive Impairment Associated with e.g. Diabetes, Alzheimer's Disease or with Parkinson's Disease

One 50 mg tablet of vildagliptin, or 20 mg of rimionabant or a combination of 50 mg of vildagliptin and 20 mg of rimionabant are administered daily with water to subjects in need of such treatment. The cognitive status of the subject is monitored periodically using the MMSE (Mini Mental State Examination (MMSE) with norms adjusted for age and education (Folstein et al., J. Psych. Res., 12 (1975), 196-198, Anthony et al., Psychological Med.' 12 (1982), 397-408; Cockrell et al., Psychopharmacology, 24 (1988), 689-692; Crum et al., J. Am. Med. Assoc'n. 18 (1993), 2386-2391) or similar tool.

### EXAMPLE 3

### Treatment for Preventing or Delaying the Onset of Alzheimer's Disease e.g. in a Subject Exhibiting Mild Cognitive Impairment

A subject having e.g. mild cognitive impairment, is identified using the MMSE or similar diagnostic tool e.g. ADAS-cog.

A 50 mg tablet of vildagliptin or 20 mg of rimionabant or a combination of 50 mg of vildagliptin and 20 mg of rimionabant are administered daily with water to said subject. The subject is monitored periodically using the MMSE or similar tool e.g. ADAS-cog, to assess the evolution of Alzheimer's Disease and the cognitive impairment. The subject is also monitored for clinical symptoms of dementia.

F. Determining the Combination Therapy Dosages.

Preferred dosages of the CB1 antagonist and DPP-IV inhibitor to be used in a combination therapy can be determined experimentally by first conducting separate dose response studies for the CB1 antagonist and DPP-IV inhibitor to be used. Methods of performing such dose response studies in a test species or the species of the intended subject (e.g., a human) are well known to one of ordinary skill in the art. The endpoint of the study is preferably selected according to the effect or endpoint of interest (e.g., appetite reduction, weight loss, body fat reduction, changes in lipid metabolism, changed food seeking behavior) Or the dose response of the underlying mechanism of action (e.g., receptor activation or antagonism). Alternatively, the established dose response relationships may be used if an agent is already well-characterized as to dose response. Preferred bioassay methods include those described above and those presented in the Examples.

### EXAMPLE 4

### The Combined Effects of CB₁ Receptor Inverse Agonist Rimonabant and DPP-4 Inhibitor e.g. Vildagliptin on Body Weight, Food Intake, Glycemia, GLP-1 Levels and Plasma Lipids in Zucker fa/fa Rats

The CB₁ receptor inverse agonist rimonabant by itself shows a reduction in body weight with a decrease in food intake, and DPP-4 inhibitor (Vildagliptin) alone improves glycemia by preserving the incretin effects. When given in combination, these two agents can interact to produce an enhanced effect on glycemia, plasma lipids and/or on body weight and food intake that can be quantified as additive or synergistic in nature.

The newly developed CB₁ receptor inverse agonist rimonabant (Sanofi-Aventis) has been shown to be highly effective to produce body weight reduction in clinic. In preclinical studies, rimonabant was effective in reversing diet-induced obesity by decreasing body weight, food intake, and by regulating circulating lipids and energy balance.

The aim of the current study is to evaluate if there is any interaction between a CB1 receptor inverse agonist e.g. rimonabant and DPP-4 inhibitors e.g. vildagliptin that can produce an improvement in the glycemic control and body weight regulation. Furthermore, the study do evaluate if there is a combined effect on improved insulin sensitivity and plasma lipids. The ‘cross talk’ between the centrally and peripherally located CB₁ receptor with the elevated endogenous incretin levels acting at the entero-insular axis can led to metabolic changes due to an interaction between the two pathways.

The current study is performed in insulin resistant male Zucker fa/fa rats. Groups of rats are treated once daily for 19 days with either vehicle, 2 doses of rimonabant alone, 1 dose of DPP-4 inhibitor Vildagliptin alone, or 2 doses of rimonabant and Vildagliptin in combination. Rats are monitored for changes in body weight, food intake, glucose tolerance, effect on GLP-1 levels, side effects such as gastrointestinal side effects and plasma lipid excursion as described below. In addition, plasma drug exposure has been measured at the end of the treatment. Alternatively, in the below experience, vildagliptin is replaced by 1-[(2-adamantyl)amino]acetyl-2-cyano-(S)-pyrrolidine (described in the patent application WO9819998A2—example 47).

Study Design and Detailed Protocol:

Study Protocol:

Animals: Ten weeks old male zucker fa/fa rats are maintained on a regular chow diet (D12492) ad ibitum from their arrival till the end of the study. Rats are housed one per cage in a reversed light cycle (9:45 am to 9:45 pm), under ACUC protocol 05 DB0062. Following a week after arrival, all rats (at 11 weeks old) are grouped as per their body weights and treated according to their group assignments (Table 1). There are two cohorts of each group that are treated 24 h apart for the entire study (cohort 1: animals #1-4, cohort 2:animals #5-8).

TABLE 1
Group assignments

| | | Rat IDs | Dose |
|---|---|---|---|
| Group | N/group | Male Zucker fa/fa | (mg/kg/day) |
| A Vehicle (1% MC) | 8 | A1-A8 | 0 |
| B Rimonabant | 8 | B1-B8 | 3 |
| C Rimonabant | 8 | C1-C8 | 10 |
| D Vildagliptin | 8 | D1-D8 | 3 |
| E Rimonabant + | 8 | E1-E8 | 3 + 3 |
| Vildagliptin | | | |
| F Rimonabant + | 8 | F1-F8 | 10 + 3 |
| Vildagliptin | | | |

Rimonabant = NVP-XSA007-AA-3(salt form): drugs are dissolved with 1% MC and preferably same is used for the combo formulation. Dosing solutions are prepared each week.

Flow Chart of the Study:

daily dosing, body weight & food intake

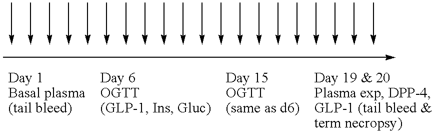

1. Preconditioning and Sorting of Rats: Day −1

Body weights and tail blood samples are taken in the morning of day −1. Blood glucose values are determined (via glucometer) and rats are sorted according to their body weight and glucose levels.

2. Dosing: Days 1 to 19

The animals are dosed orally (at 5 ml/kg) for 19 days once daily 1 h prior to the beginning of the dark cycle (before 9:45 am), with either vehicle, rimonabant or Vildagliptin alone, or in combination as shown in the Table 1.

3. Functional Tests:

a. Daily Body Weight and Food Intake Measurements:

Body weight and food intake is measured daily during the study prior to drug administration.

b. Basal levels parameters on day 1: Freely fed basal blood glucose is measured and plasma samples is collected via tail-bleed to determine insulin, glucagon, GLP-1, FFA, TG, TC, adiponectin prior to dosing on day 1.

c. Oral glucose tolerance test (OGTT) is performed on day 6 and day 15. Rats are fasted overnight, and 1-h following drug administration rats are given an oral glucose load (1 g/kg). Blood glucose and plasma samples are collected via tail-bleed at 0, 5, 10, 15, 30, 60, 120 and 180 min post-glucose load for GLP-1, insulin and glucagon measurement. In addition, at 0 min, plasma samples are collected for FFA, TG, TC measurements.

d. Plasma analysis on days 19-20: On day 19, plasma samples are collected via tail-bleed at 0 (just prior to drug dosing), 1, 2, 4, 8, and 24 h (day 20) for plasma drug concentration measurement. Plasma DPP-4 activity (5 uL) are measured at the same timepoints. Plasma GLP-1 levels are measured on day 19 at 0 (just prior to dosing), 1 h (this timepoint is right at the beginning of the food/dark cycle at 9:45 am), 1.5 h and 2 h after the drug dosing.

4. Terminal necropsy: day 20: Twenty-four hours after the last dose (day 20), plasma samples are collected via tail-bleed for glucose, insulin, GLP-1 glucagon, FFA, TG, TC, adiponectin. Following tail-bleed, rats are euthanized with CO₂ (4 min apart) and plasma samples are collected via cardiac puncture for AMP analysis (other hormonal parameters, liver and kidney safety markers in plasma). Urine is collected for analysis (AMP). Weights of liver and epididymal fat pads are taken at tissues are frozen (for TK analysis, if needed).

Results: This study do confirm the herein described unexpected effects of the herein described combinations.

Although the present invention has been described in considerable detail with reference to certain preferred versions thereof, other versions are possible without departing from the spirit and scope of the preferred versions contained herein. All references and Patents (U.S. and others) referred to herein are hereby incorporated by reference in their entirety as if set forth herein in full.

1. Combination comprising
i) a DPP IV inhibitor or a pharmaceutically acceptable salt thereof, and
ii) at least one CB1 antagonist, or a pharmaceutically acceptable salt thereof.

2. Combination according to claim 1 comprising
i) a DPP IV inhibitor or a pharmaceutically acceptable salt thereof, and
ii) at least one CB1 antagonist, or a pharmaceutically acceptable salt thereof, and at least one additional pharmaceutically acceptable carrier.

3. Combination according to claim 1, in the form of a combined preparation or a fixed combination.

4. (cancel)

5. A method for the prevention of, delay of progression of, treatment of, diseases and disorders that may be inhibited by DPP IV inhibition, appetency disorders or substance abuse disorders, comprising administering to a warm-blooded animal, including man, in need thereof a jointly effective amount of a combination of a DPP IV inhibitor or a pharmaceutically acceptable salt thereof with at least one CB1 antagonist, or a pharmaceutically acceptable salt thereof;
and at least one additional pharmaceutically acceptable carrier.

6. The method according to claim 5, wherein the disease or condition is selected from impaired glucose metabolism, conditions of impaired glucose tolerance, conditions of impaired fasting plasma glucose, diabetes particularly type 2 diabetes mellitus, obesity, diabetic retinopathy, diabetic nephropathy, diabetic neuropathy, foot ulcerations, diseases or conditions associated with diabetes, Parkinson's disease, schizophrenia, Alzheimer's disease, dementia, senile dementia, mild cognitive impairment or Alzheimer type dementia, cognitive deficits associated with schizophrenia, impaired cognitive function associated with Alzheimer's disease, impaired cognitive function associated with Parkinson's disease, appetency disorders or substance abuse disorders, or for body fat reduction.

7. The method according to claim 6, wherein the disease or condition is selected from obesity, diabetes, type 2 diabetes, IGT, Parkinson's disease, schizophrenia, Alzheimer's disease, and appetency disorders.

8. The method according to claim 5, wherein the disease or condition is selected from obesity, diabetes, IGT, type 2 diabetes, Parkinson's disease, schizophrenia, Alzheimer's disease, atherosclerosis, increased cardiovascular morbidity; excess cerebrovascular diseases; increased cardiovascular mortality and sudden death; myocardial infarction, hyperlipidemia, dyslipidemia, gastrointestinal diseases or disorders or appetency disorders.

9. A method for modulating hyperlipidemia, for modulating conditions associated with hyperlipidemia and/or for lowering VLDL, LDL and Lp(a) levels in a mammal, comprising administering to a warm-blooded animal, including man, in need thereof a jointly effective amount of a combination of a DPP IV inhibitor or a Pharmaceutically acceptable salt thereof with at least one CB1 antagonist, or a pharmaceutically acceptable salt thereof;
and at least one additional pharmaceutically acceptable carrier.

10-13. (canceled)

14. The combination according to claim 1, wherein the DPP-IV inhibitor is selected from (S)-1-{2-[5-cyanopyridin-2yl)amino]ethyl-aminoacetyl)-2-cyano-pyrrolidine, vildagliptin, Sitagliptin, GSK23A, saxagliptin, 3-(aminomethyl)-2-isobuthyl-1-oxo-4-phenyl-1,2-dihydro-6-isoquinolinecarboxamide and 2-{[3-(aminomethyl)-2-isobuthyl-4-phenyl-1-oxo-1,2-dihydro-6-isoquinolyl]oxy}acetamide, or in each case, a pharmaceutically acceptable salt thereof.

15. Combination, according to claim 1, wherein the DPP-IV inhibitor is vildagliptin or a pharmaceutically acceptable salt thereof.

16. Combination, according to claim 1, wherein the CB1 antagonist is selected from the group consisting CP-945598, rimanobant, LY-320135, AM251, AM281 and SVL326, or in each case, a pharmaceutically acceptable salt thereof.

17. Combination, according to claim 1, wherein the CB1 antagonist is rimanobant or a pharmaceutically acceptable salt thereof.

18-23. (canceled)

24. Combination, according to claim 1, wherein vildagliptin or a pharmaceutically acceptable salt thereof, is administered in an amount between 25 and 150 mg or between 50 and 100 mg daily.

25. Combination, according to claim 1, previous claims, wherein the CB1 antagonist or a pharmaceutically acceptable salt thereof, is administered in an amount between 5 and 80 mg or between 5 and 20 mg daily.

26. Combination, according to claim 16, wherein rimonabant or a pharmaceutically acceptable salt thereof, is administered in an amount between 5 and 40 mg or between 5 and 20 mg daily.

27. A combination comprising
i) vildagliptin in an amount between 25 and 150 mg or between 50 and 100 mg daily, and
ii) rimonabant in an amount between 5 and 40 mg or between 5 and 20 mg daily,
or in any case or a pharmaceutically acceptable salt thereof.

28. Combination, according to claim 27, wherein;
i) 50, 100, or 150 mg of vildagliptin is administered daily, and
ii) 5, 10 or 20 mg of rimonabant is administered daily,
or in any case or a pharmaceutically acceptable salt thereof.

29. (canceled)

30. Combination, according to claim 1, wherein the combination comprises a further antidiabetic compound, which is selected form a glitazone, metformin, and sulfonylureas.
